(19) United States
(12) Patent Application Publication
(10) Pub. No.: US 2005/0043327 A1
(43) Pub. Date: Feb. 24, 2005

(54) Pharmaceutical composition for the prevention and treatment of addiction in a mammal

(75) Inventors: Jotham Wadsworth Coe, Niantic, CT (US); Philip A. Iredale, Clinton, CT (US); Stanton Furst McHardy, Coventry, RI (US); Stafford McLean, Stonington, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/870,209

(22) Filed: Jun. 17, 2004

Related U.S. Application Data
(60) Provisional application No. 60/496,803, filed on Aug. 21, 2003.

(51) Int. Cl. A61K 31/52; A61K 31/454

(52) U.S. Cl. 514/263.22; 514/263.4; 514/326

(57) ABSTRACT

Pharmaceutical compositions are disclosed for the treatment of alcohol or cocaine dependence or addiction, tobacco dependence or addiction, reduction of alcohol withdrawal symptoms or aiding in the cessation or lessening of alcohol use or substance abuse or other behavioral dependencies including gambling. The pharmaceutical compositions are comprised of a therapeutically effective combination of an opioid receptor antagonist and a CB-1 receptor antagonist and a pharmaceutically acceptable carrier. The method of using these compounds is also disclosed.

### BACKGROUND OF THE INVENTION

The present invention relates to pharmaceutical compositions for the treatment of alcohol, cocaine or tobacco dependence or addiction in a mammal (e.g. human) comprising an opioid receptor antagonist and a CB-1 receptor antagonist. As used herein, the term “CB-1 antagonist” refers to both full antagonists and partial antagonists, as well as inverse agonists of the G-protein coupled type 1 cannabinoid receptor. For a review of cannabinoid CB1 and CB2 receptor modulators, see Pertwee, R. G., “Cannabinoid Receptor Ligands: Clinical and Neuropharmacological Considerations, Relevant to Future Drug Discovery and Development,” Exp. Opin. Invest. Drugs, 9(7), 1553-1571 (2000). The present invention may be used to treat mammals (e.g. humans) for alcohol dependence or addiction and nicotine dependence or addiction; to palliate the effects of alcohol or cocaine withdrawal, to enhance the outcomes of other alcohol cessation therapies and to treat substance abuse and behavioral dependencies, including gambling.

The compounds of the subject invention bind to opioid receptors (e.g. mu, kappa and delta opioid receptors). Compounds that bind to such receptors are likely to be useful in the treatment of diseases modulated by opioid receptors, for example irritable bowel syndrome; constipation; nausea; vomiting; and pruritic dermatoses, such as allergic dermatitis and atopy in animals and humans. Compounds that bind to opioid receptors have also been indicated in the treatment of eating disorders, opioid overdoses, depression, anxiety, schizophrenia, alcohol addiction, including alcohol abuse and dependency, sexual dysfunction, shock, stroke, spinal damage and head trauma.

The invention also relates to CB-1 receptor antagonists which include: (1) purine compounds such as those described in U.S. Provisional Application No. 60/421874, filed on Oct. 28, 2002 and incorporated herein by reference; (2) pyrazolo[1,5-a}[1,3,5]triazine compounds such as those described in U.S. Provisional Application No. 60/445728, filed on Feb. 6, 2003 and incorporated herein by reference; (3) pyrazolo[1,5-a]pyrimidine compounds such as those described in U.S. Provisional Application No. 60/446450, filed on Feb. 10, 2003 and incorporated herein by reference; (4) 1,4- and 2,4-disubstituted imidazoles such as those disclosed in U.S. Provisional Application No. 60/419621, filed on Oct. 18, 2002 and incorporated herein by reference; (5) 1-(1,5-diaryl-1H-pyrazol-3-yl)-2-(substituted amino)-ethanone compounds such as those described in U.S. Provisional Application No. 60/432911, filed on Dec. 12, 2002 and incorporated herein by reference; (6) 1-(1,5-diaryl-1H-pyrazol-3-yl)-2-(substituted amino)-ethanol compounds such as those described in U.S. Provisional Application No. 60/432911, filed on Dec. 12, 2002 and incorporated herein by reference; (7) 2-(1,5-diaryl-1H-pyrazol-3-yl)morpholine compounds such as those described in U.S. Provisional Application No. 60/432911, filed on Dec. 12, 2002 and incorporated herein by reference; and (8) 1-(1,2-diaryl-1H-imidazol-4-yl)-2-(substituted amino)-ethanone compounds such as those described in U.S. Provisional Application No. 60/432911, filed on Dec. 12, 2002 and incorporated herein by reference.

The particular opioid receptor ligands listed above, which can be employed in the methods and pharmaceutical compositions of this invention, can be made by processes known in the chemical arts, for example by the methods described in WO 03/035,622 published May 1, 2003 which is U.S. Ser. No. 10/278,142 and 60/462,651 filed Apr. 14, 2003 and 60/462,629 filed Apr. 14, 2003 and 60/462,605 filed Apr. 14, 2003 which are incorporated by reference their entireties.

Approximately 13.5 million individuals in the US suffer from alcohol abuse and dependence (AAD). Untreated alcoholics are among the highest users of US health care, consuming 15% of each health care dollar. In addition, the indirect costs associated with productivity loss, property damage, and premature death are estimated at $100 billion per year. Only 20% receive any treatment and less than 10% receive any drug treatment related to AAD. Yet it is increasingly viewed as a disease amendable to drug interventions.

### SUMMARY OF THE INVENTION

The present invention relates to a pharmaceutical composition for treating alcohol or cocaine dependence or addiction, tobacco dependence or addiction, reducing alcohol withdrawal symptoms or aiding in the cessation or lessening of alcohol use or substance abuse or behavioral dependencies including gambling, comprising:
(a) an opioid receptor antagonist or a pharmaceutically acceptable salt thereof;
(b) a CB-1 receptor antagonist or pharmaceutically acceptable salt thereof; and
(c) a pharmaceutically acceptable carrier;
wherein the active agents “a” and “b” above are present in amounts that render the composition effective in treating alcohol or cocaine dependence or addiction, tobacco dependence or addiction, reducing alcohol withdrawal symptoms or aiding in the cessation or lessening of alcohol use or substance abuse or behavioral dependencies. The therapeutically effective pharmaceutical combination is comprised of an opioid receptor antagonist and a CB-1 receptor antagonist and a pharmaceutically acceptable carrier.

U.S. Provisional Application No. 60/421874 describes CB-1 receptor antagonists purine compounds which are selected from: 1-[9-(4-chloro-phenyl)-8-(2-chlorophenyl)-9H-purin-6-yl]-3-ethylamino-azetidine-3-carboxylic acid amide; 1-[9-(4-chlorophenyl)-8-(2-chlorophenyl)-9H-purin-6-yl]-3-isopropylaminoazetidine-3-carboxylic acid amide; 1-{1-[9-(4-chlorophenyl)-8-(2-chlorophenyl)-9H-purin-6-yl]-4-phenylpiperidin-4-yl}-ethanone; {3-[9-(4-chlorophenyl)-8-(2,4-dichlorophenyl)-9H-purin-6-yl]-3-(1α,5α,6α)-azabicyclo[3.1.0]hex-6-yl}-dimethylamine; 6-1-benzylpyrrolidin-3-yloxy)-9-(4-chlorophenyl)-8-(2,4-dichlorophenyl)-9H-purine; 9-(4chlorophenyl)-6-(1-cyclohexylazetidin-3-yloxy)-8-(2,4-dichlorophenyl)-9H-purine; 6-tert-butoxy-9-(4-chlorophenyl)-8-(2,4-dichlorophenyl)-9H-purine; 9-(4-chlorophenyl)-8-(2,4-dichlorophenyl)-6-isopropoxy-9H-purine; 1-[9-(4-chlorophenyl)-8-(2,4-dichlorophenyl)-9H-purin-6-yl]-4-propylaminopiperidine-4-carboxylic acid amide; 1-[9-(4-chlorophenyl)-8-(2-fluorophenyl)-9H-purin-6-yl]-4-propylaminopiperidine-4-carboxylic acid amide; 1-[9-(4-chlorophenyl)-8-(2-chlorophenyl)-9H-purin-6-yl]-4-propylaminopiperidine-4-carboxylic acid amide; 1-[9-(4-chlorophenyl)-8-(2-fluorophenyl)-2-methyl-9H-purin-6-yl]-4-isopropylaminopiperidine-4-carboxylic acid amide; 1-[9-(4-chlorophenyl)-8-(2-chlorophenyl)-9H-purin-6-yl]-4-pyrrolidin-1-yl-piperidine-4-carboxylic acid amide; 1-[9-(4-chlorophenyl)-8-(2-chlorophenyl)-9H-purin-6-yl]-4-ethylamino-piperidine-4-carboxylic acid amide; 1-[9-(4-chlorophenyl)-8-(2-chlorophenyl)-9H-purin-6-yl]-4-isopropylaminopiperidine-4-carboxylic acid amide; 4-amino-1-[9-(4-chlorophenyl)-8-(2-chlorophenyl)-9H-purin-6-yl]-piperidine-4-carboxylic acid amide; 1-[9-(4-chlorophenyl)-8-(2,4-dichlorophenyl)-9H-purin-6-yl]-4-methylaminopiperidine-4-carboxylic acid amide; 1-[9-(4-chlorophenyl)-8-(2-fluorophenyl)-9H-purin-6-yl]-4-isopropylaminopiperidine-4-carboxylic acid amide; 8-[9-(4-chlorophenyl)-8-(2-chlorophenyl)-9H-purin-6-yl]-1-isopropyl-1,3,8-triazaspiro[4.5]decan-4-one; 9-[9-(4-chlorophenyl)-8-(2-chlorophenyl)-9H-purin-6-yl]-1-methyl-4-oxa-1,9-diazaspiro[5.5]undecan-2-one; 8-[9-(4-chlorophenyl)-8-(2,4-dichlorophenyl)-9H-purin-6-yl]-1-isopropyl-1,3,8-triazaspiro[4.5]decan-4-one; 1-[9-(4-chlorophenyl)-8-(2-chlorophenyl)-9H-purin-6-yl]-4-(4-fluorophenyl)-piperidin-4-ol; 1-[9-(4-chlorophenyl)-8-(2-chlorophenyl)-9H-purin-6-yl]-4-phenylpiperid in-4-ol; 4-benzyl-1-[9-(4-chlorophenyl)-8-(2-chlorophenyl)-9H-purin-6-yl]-piperidin-4-ol; 4-[9-(4-chlorophenyl)-8-(2-chlorophenyl)-9H-purin-6-yl]-piperazine-2-carboxylic acid methylamide; 9-(4-chlorophenyl)-8-(2,4-dichlorophenyl)-6-(4-pyridin-2-yl-piperazin-1-yl)-9H-purine; and 9-(4-chlorophenyl)-8-(2,4-dichlorophenyl)-6-(4-pyrimidin-2-yl-piperazin-1-yl)-9H-purine; 1-[9-(4-chlorophenyl)-8-(2-fluorophenyl)-9H-purin-6-yl]-4-isopropylaminopiperidine-4-carboxylic acid amide; 1-[9-(4-chlorophenyl)-8-(2-chlorophenyl)-9H-purin-6-yl]-4-isopropylamino-piperidine-4-carboxylic acid amide; 4-amino-1-[9-(4-chlorophenyl)-8-(2-chlorophenyl)-9H-purin-6-yl]-piperidine-4-carboxylic acid amide; 1-[9-(4-chlorophenyl)-8-(2-chlorophenyl)-9H-purin-6-yl]-4-ethylamino-piperidine-4-carboxylic acid amide; 8-[9-(4-chlorophenyl)-8-(2-chlorophenyl)-9H-purin-6-yl]-1-isopropyl-1,3,8-triazaspiro[4.5]decan-4-one; 4-amino-1-[9-(4-chloro-phenyl)-8-(2-chlorophenyl)-9H-purin-6-yl]-piperidine-4-carboxylic acid amide; and 1-[9-(4-chlorophenyl)-8-(2-chlorophenyl)-9H-purin-6-yl]-4-ethylaminopiperidine-4-carboxylic acid amide; and a pharmaceutically acceptable salt thereof or a solvate or hydrate of said compound or said salt.

U.S. Provisional Application No. 60/445728 describes CB-1 receptor antagonist pyrazolo[1,5-a}[1,3,5]triazine compounds selected from: 7-(2-chlorophenyl)-8-(4-chlorophenyl)-2-methyl-4-(4-methylpiperazin-1-yl)-pyrazolo[1,5-a][1,3,5]triazine; 7-(2-chlorophenyl)-8-(4-chlorophenyl)-2-methyl-4-(4-pyrimidin-2-ylpiperazin-1-yl)-pyrazolo[1,5-a][1,3,5]triazine; 7-(2-chlorophenyl)-8-(4-chlorophenyl)-4-[(1S,4S)-5-methanesulfonyl-2,5-diazabicyclo[2.2.1]hept-2-yl]-2-methylpyrazolo[1,5-a][1,3,5]triazine; and 7-(2-chlorophenyl)-8-(4-chlorophenyl)-2-methyl-4-[4-(propane-2-sulfonyl)-piperazin-1-yl]-pyrazolo[1,5-a][1,3,5]triazine; 1-[7-(2-chlorophenyl)-8-(4-chlorophenyl)-2-methylpyrazolo[1,5-a][1,3,5]triazin-4-yl]-4-methylaminopiperidine-4-carboxylic acid amide; 1-[7-(2-chlorophenyl)-8-(4-fluorophenyl)-2-methylpyrazolo[1,5-a][1,3,5]triazin-4-yl]-4-ethylaminopiperidine-4-carboxylic acid amide; 1-[7-(2-chlorophenyl)-8-(4-chlorophenyl)-2-methylpyrazolo[1,5-a][1,3,5]triazin-4-yl]-4-ethylaminopiperidine-4-carboxylic acid amide; 1-[7-(2-chlorophenyl)-8-(4-chlorophenyl)-2-methylpyrazolo[1,5-a][1,3,5]triazin-4-yl]-4-isopropylaminopiperidine-4-carboxylic acid amide; 1-[7-(2-chlorophenyl)-8-(4-chlorophenyl)-2-methylpyrazolo[1,5-a][1,3,5]triazin-4-yl]-3-ethylaminoazetidine-3-carboxylic acid amide; 1-[7-(2-chlorophenyl)-8-(4-chlorophenyl)-2-methylpyrazolo[1,5-a][1,3,5]triazin-4-yl]-3-isopropylaminoazetidine-3-carboxylic acid amide; 3-amino-1-[7-(2-chlorophenyl)-8-(4-chlorophenyl)-2-methylpyrazolo[1,5-a][1,3,5]triazin-4-yl]-azetidine-3-carboxylic acid amide; 1-[7-(2-chlorophenyl)-8-(4-chlorophenyl)-2-methylpyrazolo[1,5-a][1,3,5]triazin-4-yl]-3-methylaminoazetidine-3-carboxylic acid amide; and 1-[7-(2-chlorophenyl )-8-(4-chlorophenyl)-2-methylpyrazolo[1,5-a][1,3,5]triazin-4-yl]-3-dimethylaminoazetidine-3-carboxylic acid amide; 1-{1-[7-(2-chlorophenyl)-8-(4-chlorophenyl)-2-methylpyrazolo[1,5-a][1,3,5]triazin-4-yl]-4-phenylpiperidin-4-yl}-ethanone; 3-[7-(2-chlorophenyl)-8-(4-chlorophenyl)-2-methylpyrazolo[1,5-a][1,3,5]triazin-4-yl]-3-azabicyclo[3.1.0]hex-6-ylamine; 1-[7-(2-chlorophenyl)-8-(4-chlorophenyl )-2-methylpyrazolo[1,5-a][1,3,5]triazin-4-yl]-4-(4-fluorophenyl)-piperidin-4-ol; 4-benzyl-1-[7-(2-chlorophenyl)-8-(4-chlorophenyl)-2-methylpyrazolo[1,5-a][1,3,5]triazin-4-yl]-piperidin-4-ol; 2-[7-(2-chlorophenyl)-8-(4-chlorophenyl )-2-methylpyrazolo[1,5-a][1,3,5]triazin-4-yl]-5-methyl-2,5,7-triazaspiro[3.4]octan-8-one; 2-[7-(2-chlorophenyl)-8-(4-chlorophenyl)-2-methylpyrazolo[1,5-a][1,3,5]triazin-4-yl]-2,5,7-triazaspiro[3.4]octan-8-one; 8-[7-(2-chlorophenyl)-8-(4-chlorophenyl)-2-methylpyrazolo[1,5-a][1,3,5]triazin-4-yl]-1-isopropyl-1,3,8-triazaspiro[4.5]decan-4-one; 2-[7-(2-chlorophenyl)-8-(4-chlorophenyl)-2-methylpyrazolo[1,5-a][1,3,5]triazin-4-yl]-6,6-dimethyl-2,5,7-triazaspiro[3.4]octan-8-one; 4-(1-benzylpyrrolidin-3-yloxy)-7-(2-chlorophenyl)-8-(4-chlorophenyl)-2-methylpyrazolo[1,5-a][1,3,5]triazine; 7-(2-chlorophenyl)-8-(4-chlorophenyl)-4-(1-cyclohexylazetidin-3-yloxy)-2-methylpyrazolo[1,5-a][1,3,5]triazine; 7-(2-chlorophenyl)-8-(4-chlorophenyl)-4-isopropoxy-2-methylpyrazolo[1,5-a][1,3,5]triazine; and 4-tert-butoxy-7-(2-chlorophenyl)-8-(4-chlorophenyl)-2-methylpyrazolo[1,5-a][1,3,5]triazine; butyl-[7-(2-chlorophenyl)-8-(4-chlorophenyl)-2-methylpyrazolo[1,5-a][1,3,5]triazin-4-yl]-amine; 7-(2-chlorophenyl)-8-(4-chlorophenyl)-2-methyl-4-piperidin-1-yl-pyrazolo[1,5-a][1,3,5]triazine; [7-(2-chlorophenyl)-8-(4-chlorophenyl)-2-methylpyrazolo[1,5-a][1,3,5]triazin-4-yl]-[2-(4-fluorophenyl)-ethyl]-amine; 7-(2-chlorophenyl)-8-(4-chlorophenyl)-2-methyl-4-morpholin-4-yl-pyrazolo[1,5-a][1,3,5]triazine; and [7-(2-chlorophenyl)-8-(4-chlorophenyl)-2-methylpyrazolo[1,5-a][1,3,5]triazin-4-yl]-(2-morpholin-4-yl-ethyl)-amine; 1-[7-(2-chlorophenyl)-8-(4-chlorophenyl)-2-methylpyrazolo[1,5-a][1,3,5]triazin-4-yl]-4-ethylaminopiperidine-4-carboxylic acid amide; 1-[7-(2-chlorophenyl)-8-(4-chlorophenyl)-2-methylpyrazolo[1,5-a][1,3,5]triazin-4-yl]-3-ethylaminoazetidine-3-carboxylic acid amide; 1-[7-(2-chlorophenyl)-8-(4-chlorophenyl)-2-methylpyrazolo[1,5-a][1,3,5]triazin-4-yl]-3-isopropylaminoazetidine-3-carboxylic acid amide; 3-amino-1-[7-(2-chlorophenyl)-8-(4-chlorophenyl)-2-methylpyrazolo[1,5-a][1,3,5]triazin-4-yl]-azetidine-3-carboxylic acid amide; and 8-[7-(2-chlorophenyl)-8-(4-chlorophenyl)-2-methylpyrazolo[1,5-a][1,3,5]triazin-4-yl]-1-isopropyl-1,3,8-triazaspiro[4.5]decan-4-one; and a pharmaceutically acceptable salt thereof or a solvate or hydrate of said compound or said salt.

U.S. Provisional Application No. 60/446450 describes CB-1 receptor antogonist pyrazolo[1,5-a]pyrimidine compounds selected from: 3-(4-chlorophenyl)-2-(2-chlorophenyl)-7-(4-methyl-piperazin-1-yl)-pyrazolo[1,5-a]pyrimidine; 3-(4-chlorophenyl)-2-(2-chlorophenyl)-7-(4-pyrimidin-2-yl-piperazin-1-yl)-pyrazolo[1,5-a]pyrim idine; 3-(4-chloro-phenyl)-2-(2-chlorophenyl)-7-[(1S,4S)-5-methanesulfonyl-2,5-diazabicyclo[2.2.1]hept-2-yl]-pyrazolo[1,5-a]pyrimidine; and 3-(4-chlorophenyl)-2-(2-chlorophenyl)-7-[4-(propane-2-sulfonyl)-piperazin-1-yl]-pyrazolo[1,5-a]pyrimidine; 1-[3-(4-chlorophenyl)-2-(2-chlorophenyl)-pyrazolo[1,5-a]pyrimidin-7-yl]-4-ethylaminopiperidine-4-carboxylic acid amide; 1-[3-(4-chlorophenyl)-2-(2-chlorophenyl)-pyrazolo[1,5-a]pyrimidin-7-yl]-4-isopropylaminopiperidine-4-carboxylic acid amide; 1-[3-(4-chlorophenyl)-2-(2-chlorophenyl)-pyrazolo[1,5-a]pyrimidin-7-yl]-3-ethylaminoazetidine-3-carboxylic acid amide; 3-amino-1-[3-(4-chlorophenyl)-2-(2-chlorophenyl)-pyrazolo[1,5-a]pyrimidin-7-yl]-azetidine-3-carboxylic acid amide; 1-[3-(4-chlorophenyl)-2-(2-chlorophenyl)-6-methylpyrazolo[1,5-a]pyrimidin-7-yl]-3-ethylaminoazetidine-3-carboxylic acid amide; 1-[3-(4-chlorophenyl)-2-(2-chlorophenyl)-pyrazolo[1,5-a]pyrimidin-7-yl]-3-isopropylaminoazetidine-3-carboxylic acid amide; 1-[3-(4-chlorophenyl)-2-(2-chlorophenyl)-5,6-dimethylpyrazolo[1,5-a]pyrimidin-7-yl]-3-ethylamino-azetidine-3-carboxylic acid amide; 1-[3-(4-chlorophenyl)-2-(2-chlorophenyl)-pyrazolo[1,5-a]pyrimidin-7-yl]-3-methylaminoazetidine-3-carboxylic acid amide; 1-[3-(4-chlorophenyl)-2-(2-chlorophenyl)-5-methylpyrazolo[1,5-a]pyrimidin-7-yl]-3-ethylaminoazetidine-3-carboxylic acid amide; 1-{1-[3-(4-chlorophenyl)-2-(2-chlorophenyl)-pyrazolo[1,5-a]pyrimidin-7-yl]-4-phenylpiperidin-4-yl}-ethanone; 3-[3-(4-chlorophenyl)-2-(2-chlorophenyl)-pyrazolo[1,5-a]pyrimidin-7-yl]-3-(1a,5a,6a)-azabicyclo[3.1.0]hex-6-ylamine; 1-[3-(4-chlorophenyl)-2-(2-chlorophenyl)-pyrazolo[1,5-a]pyrimidin-7-yl]-4-(4-fluorophenyl)-piperidin-4-ol; 4-benzyl-1-[3-(4-chlorophenyl)-2-(2-chlorophenyl)-pyrazolo[1,5-a]pyrimidin-7-yl]-piperidin-4-ol; 8-[3-(4-chlorophenyl)-2-(2-chlorophenyl)-pyrazolo[1,5-a]pyrimidin-7-yl]-1-isopropyl-1,3,8-triazaspiro[4.5]decan-4-one; 2-[3-(4-chlorophenyl)-2-(2-chlorophenyl)-pyrazolo[1,5-a]pyrimidin-7-yl]-2,5,7-triazaspiro[3.4]octan-8-one; 8-[3-(4-chlorophenyl)-2-(2-chlorophenyl)-6-methylpyrazolo[1,5-a]pyrimidin-7-yl]-1-isopropyl-1,3,8-triazaspiro[4.5]decan-4-one; 2-[3-(4-chlorophenyl)-2-(2-chlorophenyl)-pyrazolo[1,5-a]pyrimidin-7-yl]-5-methyl-2,5,7-triazaspiro[3.4]octan-8-one; 7-(1-benzylpyrrolidin-3-yloxy)-3-(4-chlorophenyl)-2-(2-chlorophenyl)-pyrazolo[1,5-a]pyrimidine; and 3-(4-chlorophenyl)-2-(2-chlorophenyl)-7-(1-cyclohexylazetidin-3-yloxy)-pyrazolo[1,5-a]pyrimidine; 1-[3-(4-chlorophenyl)-2-(2-chlorophenyl)-pyrazolo[1,5-a]pyrimidin-7-yl]-4-ethylaminopiperidine-4-carboxylic acid amide; 1-[3-(4-chlorophenyl)-2-(2-chlorophenyl)-pyrazolo[1,5-a]pyrimidin-7-yl]-4-isopropylaminopiperidine-4-carboxylic acid amide; and 1-[3-(4-chlorophenyl)-2-(2-chlorophenyl)-pyrazolo[1,5-a]pyrimidin-7-yl]-3-ethylaminoazetidine-3-carboxylic acid amide; 8-[3-(4-chlorophenyl)-2-(2-chlorophenyl)-pyrazolo[1,5-a]pyrimidin-7-yl]-1-isopropyl-1,3,8-triazaspiro[4.5]decan-4-one; and a pharmaceutically acceptable salt thereof or a solvate or hydrate of said compound or said salt.

U.S. Provisional Application No. 60/419621 describes CB-1 receptor antagonist 1,4- and 2,4-disubstituted imidazoles selected from: 5-(4-chloro-phenyl)-3-(5-cyclohexyl-1H-imidazol-2-yl)-1-(2,4-dichloro-phenyl)-4-methyl-1H-pyrazole; 5-(4-chloro-phenyl)-3-(2-cyclohexyl-3H-imidazol-4-yl)-1-(2,4-dichloro-phenyl)-4-methyl-1H-pyrazole; 5-(4-chloro-phenyl)-1 -(2,4-dichloro-phenyl)-4-methyl-3-[1-(1-methyl-1-phenyl-ethyl)-1H-imidazol-4-yl]-1H-pyrazole; 5-(4-chloro-phenyl)-1-(2-chloro-phenyl)-4-methyl-3-[1-(1-phenyl-ethyl)-1H-imidazol-4-yl]-1H-pyrazole; 5-(4-chloro-phenyl)-1-(2-fluoro-phenyl)-4-methyl-3-[1-(1-methyl-1-phenyl-ethyl)-1H-imidazol-4-yl]-1H-pyrazole; 5-(4-chloro-phenyl)-1-(2-chloro-phenyl)-3-[1-(2,2-dimethyl-tetrahydro-pyran-4-yl)-1H-imidazol-4-yl]-4-methyl-1H-pyrazole: 5-{2-(2,4-dichloro-phenyl)-4-methyl-5-[1-(1-methyl-1-phenyl-ethyl)-1H-imidazol-4-yl]-2H-pyrazol-3-yl}-2-methoxy-pyridine; and 1-(2-chloro-phenyl)-5-(4-chloro-phenyl)-4-methyl-3-[1-(1-methyl-1-phenyl-ethyl)-1H-imidazol-4-yl]-1H-pyrazole; and a pharmaceutically acceptable salt thereof or a solvate or hydrate of the compound or the salt.

U.S. Provisional Application No. 60/432911 describes CB-1 receptor antagonist 1-(1,5-diaryl-1H-pyrazol-3-yl)-2-(substituted amino)-ethanol compounds selected from: 1-[5-(4-chloro-phenyl)-1-(2-chloro-phenyl)-4-methyl-1H-pyrazol-3-yl]-2-piperidin-1-yl-ethanone; 1-[5-(4-chloro-phenyl)-1-(2-chloro-phenyl)-4-methyl-1H-pyrazol-3-yl]-2-morpholin-4-yl-ethanone; 1-[5-(4-chloro-phenyl)-1-(2-chloro-phenyl)-4-methyl-1H-pyrazol-3-yl]-2-[4-(1-methyl-1H-pyrrole-2-carbonyl)-piperazin-1-yl]-ethanone; 1-[5-(4-chloro-phenyl)-1-(2-chloro-phenyl)-4-methyl-1H-pyrazol-3-yl]-2-[4-(1-methyl-cyclopropanecarbonyl)-piperazin-1-yl]-ethanone; N-(1-{2-[5-(4-chloro-phenyl)-1-(2-chloro-phenyl)-4-methyl-1H-pyrazol-3-yl]-2-oxo-ethyl}-piperidin-4-yl)-2,2,2-trifluoro-acetamide; 1-[5-(4-chloro-phenyl)-1-(2-fluoro-phenyl)-4-methyl-1H-pyrazol-3-yl]-2-morpholin-4-yl-ethanone; 1-[5-(4-chloro-phenyl)-1-(2-fluoro-phenyl)-4-methyl-1H-pyrazol-3-yl]-2-piperidin-1-yl-ethanone; 1-[5-(4-chloro-phenyl)-1-(2-fluoro-phenyl)-4-methyl-1H-pyrazol-3-yl]-2-(4-trifluoroacetyl-piperazin-1-yl)-ethanone; 1-[1-(2-chloro-phenyl)-5-(4-chloro-phenyl)-4-methyl-1H-pyrazol-3-yl]-2-pyrrolidin-1-yl-ethanone; 1 -[1 -(2-chloro-phenyl)-5-(4-chloro-phenyl)-4-methyl-1H-pyrazol-3-yl]-2-[1,4]oxazepan-4-yl-ethanone; and 1-[5-(4-chloro-phenyl)-1-(2-chloro-phenyl)-4-methyl-1H-pyrazol-3-yl]-2-(1-oxa-8-aza-spiro[4.5]dec-8-yl)-ethanone; and a pharmaceutically acceptable salt thereof, or a solvate or hydrate of the compound or the salt.

U.S. Provisional Application No. 60/432911 describes CB-1 receptor antagonist 1-(1,5-diaryl-1H-pyrazol-3-yl)-2-(substituted amino)-ethanol compounds selected from: 2-(benzyl-isopropyl-amino)-1-[1-(2-chloro-phenyl)-5-(4-chloro-phenyl)-4-methyl-1H-pyrazol-3-yl]-ethanol; 1-[5-(4-chloro-phenyl)-1-(2-chloro-phenyl)-4-methyl-1H-pyrazol-3-yl]-2-(3,5-dimethyl-piperidin-1-yl)-ethanol; 1-{2-[1-(2-chloro-phenyl)-5-(4-chloro-phenyl)-4-methyl-1H-pyrazol-3-yl]-2-hydroxy-ethyl}-4-isopropylamino-piperidine-4-carboxylic acid amide; 1-[5-(4-chloro-phenyl)-1-(2-chloro-phenyl)-4-methyl-1H-pyrazol-3-yl]-2-(3,3-dimethyl-piperidin-1-yl)-ethanol; 1-[5-(4-chloro-phenyl)-1-(2-chloro-phenyl)-4-methyl-1H-pyrazol-3-yl]-2-piperidin-1-yl-ethanol; and 1-[5-(4-chloro-phenyl)-1-(2-chloro-phenyl)-4-methyl-1H-pyrazol-3-yl]-2-morpholin-4-yl-ethanol; and a pharmaceutically acceptable salt thereof, or a solvate or hydrate of the compound or the salt.

U.S. Provisional Application No. 60/432911 describes CB-1 receptor antagonist 1-(1,2-diaryl-1H-imidazol-4-yl)-2-(substituted amino)-ethanone compounds selected from: 1-[1-(4-chloro-phenyl)-2-(2,4-dichloro-phenyl)-5-methyl-1H-imidazol-4-yl]-2-piperidin-1-yl-ethanone and 1-[1-(4-chloro-phenyl )-2-(2,4-dichloro-phenyl )-5-methyl- 1H-imidazol-4-yl]-2-morpholin-4-yl-ethanone; and a pharmaceutically acceptable salt thereof, or a solvate or hydrate of the compound or the salt.

In another more specific embodiment of this invention, the opioid receptor antagonist is selected from:
2-methoxy-ethanesulfonic acid {3-[6-ethyl-3-(2-hydroxy-1,2,3,4-tetrahydro-naphthalen-2-ylmethyl)-3-aza-bicyclo[3.1.0]hex-6-yl]-phenyl}-amide;
N-[3-(6-ethyl-3-indan-2-ylmethyl-3-aza-bicyclo[3.1.0]hex-6-yl)-phenyl]-methanesulfonamide;
2-methoxy-ethanesulfonic acid [3-(6-ethyl-3-indan-2-yl methyl-3-aza-bicyclo[3.1.0]hex-6-yl)-phenyl]-amide;
N-{3-[6-ethyl-3-(2-hydroxy-indan-2-ylmethyl)-3-aza-bicyclo[3.1.0]hex-6-yl]-phenyl}-methanesulfonamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
N-(3-{6-ethyl-3-[3-(1-hydroxy-cyclohexyl )-propyl]-3-aza-bicyclo[3.1.0]hex-6-yl}-phenyl)-methanesulfonamide;
3-{3-[3-(1-hydroxy-cyclohexyl)-propyl]-8-methoxy-3-aza-bicyclo[3.2.1]oct-8-yl}-benzamide;
2-methoxy-ethanesulfonic acid {3-[6-ethyl-3-(2-hydroxy-indan-2-ylmethyl)-3-aza-bicyclo[3.1.0]hex-6-yl]-phenyl}-amide;
3-[6-ethyl-3-(2-hydroxy-indan-2-ylmethyl)-3-aza-bicyclo[3.1.0]hex-6-yl]-benzamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
3-[6-ethyl-3-(2-hydroxy-1,2,3,4-tetrahydro-naphthalen-2-ylmethyl)-3-aza-bicyclo[3.1.0]hex-6-yl]-benzamide;
N-{3-[2-(2-hydroxy-indan-2-ylmethyl)-2-aza-bicyclo[3.3.1]non-5-yl]-phenyl}-methanesulfonamide;
2-methoxy-ethanesulfonic acid {3-[3-(2-hydroxy-indan-2-ylmethyl)-8-methoxy-3-aza-bicyclo[3.2.1]oct-8-yl]-phenyl}-amide;
3-{6-ethyl-3-[3-(1-hydroxy-cyclohexyl)-propyl]-3-aza-bicyclo[3.1.0]hex-6-yl}-benzamide;
3-[6-ethyl-3-(1-hydroxy-3-phenyl-cyclobutylmethyl)-3-aza-bicyclo[3.1.0]hex-6-yl]-benzamide;
2-methoxy-ethanesulfonic acid (3-{6-ethyl-3-[3-(1-hydroxy-cyclohexyl)-propyl]-3-aza-bicyclo[3.1.0]hex-6-yl}-phenyl)-amide;
3-{1 -[3-(1-hydroxy-cyclohexyl)-propyl]-3,4-dimethyl-piperidin-4-yl}-benzamide;
3-(1-indan-2-ylmethyl-3,4-dimethyl-piperidin-4-yl)-benzamide;
N-{3-[3-(2-hydroxy-indan-2-ylmethyl)-8-methoxy-3-aza-bicyclo[3.2.1]oct-8-yl]-phenyl}-methanesulfonamide;
3-[1-(1-hydroxy-3-phenyl-cyclobutylmethyl)-3,4-dimethyl-piperidin-4-yl]-benzamide;
3-(6-ethyl-3-indan-2-ylmethyl-3-aza-bicyclo[3.1.0]hex-6-yl)-benzamide;
N-(3-{3-[3-(1-hydroxy-cyclohexyl)-propyl]-8-methoxy-3-aza-bicyclo[3.2.1]oct-8-yl}-phenyl)-methanesulfonamide;
3-[1-(2-hydroxy-indan-2-ylmethyl)-3,4-dimethyl-piperidin-4-yl]-benzamide;
N-(3-{2-[3-(1-hydroxy-cyclohexyl)-propyl]-2-aza-bicyclo[3.3.1]non-5-yl}-phenyl)-methanesulfonamide;
3-[3-(1-hydroxy-3-phenyl-cyclobutylmethyl)-8-methoxy-3-aza-bicyclo[3.2.1]oct-8-yl]-benzamide;
3-[3-(2-hydroxy-indan-2-ylmethyl)-8-methoxy-3-aza-bicyclo[3.2.1]oct-8-yl]-benzamide;
3-[2-(2-hydroxy-indan-2-ylmethyl)-2-aza-bicyclo[3.3.1]non-5-yl]-benzamide;
3-{2-[3-(1-hydroxy-cyclohexyl)-propyl]-2-aza-bicyclo[3.3.1]non-5-yl}-benzamide;
3-[2-(1 -hydroxy-3-phenyl-cyclobutylmethyl)-2-aza-bicyclo[3.3.1]non-5-yl]-benzamide;
3-[2-(2-hydroxy-1,2,3,4-tetrahydro-naphthalen-2-ylmethyl)-2-aza-bicyclo[3.3.1]non-5-yl]-benzamide;
2-methoxy-ethanesulfonic acid {3-[2-(2-hydroxy-indan-2-ylmethyl)-2-aza-bicyclo[3.3.1]non-5-yl]-phenyl}-amide;
2-methoxy-ethanesulfonic acid (3-{2-[3-(1-hydroxy-cyclohexyl)-propyl]-2-aza-bicyclo[3.3.1]non-5-yl}-phenyl)-amide;
2-methoxy-ethanesulfonic acid {3-[2-(1-hydroxy-3-phenyl-cyclobutylmethyl)-2-aza-bicyclo[3.3.1]non-5-yl]-phenyl}-amide;
2-methoxy-ethanesulfonic acid {3-[2-(2-hydroxy-1,2,3,4-tetrahydro-naphthalen-2-ylmethyl)-2-aza-bicyclo[3.3.1]non-5-yl]-phenyl}-amide;
N-{3-[2-(1-hydroxy-3-phenyl-cyclobutylmethyl)-2-aza-bicyclo[3.3.1]non-5-yl]-phenyl}-methanesulfonamide; and
N-{3-[2-(2-hydroxy-1,2,3,4-tetrahydro-naphthalen-2-ylmethyl)-2-aza-bicyclo[3.3.1]non-5-yl]-phenyl}-methanesulfonamide.

Preferably, the opioid receptor antagonist is selected from:
2-methoxy-ethanesulfonic acid {3-[6-ethyl-3-(2-hydroxy-1,2,3,4-tetrahydro-naphthalen-2-ylmethyl )-3-aza-bicyclo[3.1.0]hex-6-yl]-phenyl}-amide;
N-[3-(6-ethyl-3-indan-2-ylmethyl-3-aza-bicyclo[3.1.0]hex-6-yl)-phenyl]-methanesulfonamide;
2-methoxy-ethanesulfonic acid [3-(6-ethyl-3-indan-2-yl methyl-3-aza-bicyclo[3.1.0]hex-6-yl)-phenyl]-amide;
N-{3-[6-ethyl-3-(2-hydroxy-indan-2-ylmethyl )-3-aza-bicyclo[3.1.0]hex-6-yl]-phenyl}-methanesulfonamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
N-(3-{6-ethyl-3-[3-(1-hydroxy-cyclohexyl)-propyl]-3-aza-bicyclo[3.1.0]hex-6-yl}-phenyl)-methanesulfonamide;
3-{3-[3-(1-hydroxy-cyclohexyl)-propyl]-8-methoxy-3-aza-bicyclo[3.2.1]oct-8-yl}-benzamide;
2-methoxy-ethanesulfonic acid {3-[6-ethyl-3-(2-hydroxy-indan-2-ylmethyl)-3-aza-bicyclo[3.1.0]hex-6-yl]-phenyl}-amide;
3-[6-ethyl-3-(2-hydroxy-indan-2-ylmethyl)-3-aza-bicyclo[3.1.0]hex-6-yl]-benzamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
3-[6-ethyl-3-(2-hydroxy-1,2,3,4-tetrahydro-naphthalen-2-ylmethyl )-3-aza-bicyclo[3.1.0]hex-6-yl]-benzamide;
N-{3-[2-(2-hydroxy-indan-2-ylmethyl)-2-aza-bicyclo[3.3.1]non-5-yl]-phenyl}-methanesulfonamide;
2-methoxy-ethanesulfonic acid {3-[3-(2-hydroxy-indan-2-ylmethyl)-8-methoxy-3-aza-bicyclo[3.2.1]oct-8-yl]-phenyl}-amide;
3-{6-ethyl-3-[3-(1-hydroxy-cyclohexyl)-propyl]-3-aza-bicyclo[3.1.0]hex-6-yl}-benzamide;
3-[6-ethyl-3-(1-hydroxy-3-phenyl-cyclobutylmethyl)-3-aza-bicyclo[3.1.0]hex-6-yl]-benzamide;
2-methoxy-ethanesulfonic acid (3-{6-ethyl-3-[3-(1-hydroxy-cyclohexyl)-propyl]-3-aza-bicyclo[3.1.0]hex-6-yl}-phenyl)-amide;
3-{1-[3-(1-hydroxy-cyclohexyl)-propyl]-3,4-dimethyl-piperidin-4-yl}-benzamide;
3-(1 -indan-2-ylmethyl-3,4-dimethyl-piperidin-4-yl)-benzamide;
N-{3-[3-(2-hydroxy-indan-2-ylmethyl)-8-methoxy-3-aza-bicyclo[3.2.1]oct-8-yl]-phenyl}-methanesulfonamide;
3-[1-(1-hydroxy-3-phenyl-cyclobutylmethyl)-3,4-dimethyl-piperidin-4-yl]-benzamide;
3-(6-hthyl-3-indan-2-ylmethyl-3-aza-bicyclo[3.1.0]hex-6-yl)-benzamide;
N-(3-{3-[3-(1-hydroxy-cyclohexyl)-propyl]-8-methoxy-3-aza-bicyclo[3.2.1]oct-8-yl}-phenyl)-methanesulfonamide;
3-[1 -(2-hydroxy-indan-2-ylmethyl)-3,4-dimethyl-piperidin-4-yl]-benzamide;
N-(3-{2-[3-(1-hydroxy-cyclohexyl)-propyl]-2-aza-bicyclo[3.3.1]non-5-yl}-phenyl)-methanesulfonamide;
3-[3-(1-hydroxy-3-phenyl-cyclobutylmethyl)-8-methoxy-3-aza-bicyclo[3.2.1]oct-8-yl]-benzamide;
3-[3-(2-hydroxy-indan-2-ylmethyl)-8-methoxy-3-aza-bicyclo[3.2.1]oct-8-yl]-benzamide;
3-[2-(2-hydroxy-indan-2-yl methyl)-2-aza-bicyclo[3.3.1]non-5-yl]-benzamide;
3-{2-[3-(1-hydroxy-cyclohexyl)-propyl]-2-aza-bicyclo[3.3.1]non-5-yl}-benzamide;
3-[2-(1-hydroxy-3-phenyl-cyclobutylmethyl)-2-aza-bicyclo[3.3.1]non-5-yl]-benzamide;
3-[2-(2-hydroxy-1,2,3,4-tetrahydro-naphthalen-2-ylmethyl)-2-aza-bicyclo[3.3.1]non-5-yl]-benzamide;
2-methoxy-ethanesulfonic acid {3-[2-(2-hydroxy-indan-2-ylmethyl)-2-aza-bicyclo[3.3.1]non-5-yl]-phenyl}-amide;
2-methoxy-ethanesulfonic acid (3-{2-[3-(1-hydroxy-cyclohexyl)-propyl]-2-aza-bicyclo[3.3.1]non-5-yl}-phenyl)-amide;
2-methoxy-ethanesulfonic acid {3-[2-(1-hydroxy-3-phenyl-cyclobutylmethyl)-2-aza-bicyclo[3.3.1]non-5-yl]-phenyl}-amide;
2-methoxy-ethanesulfonic acid {3-[2-(2-hydroxy-1,2,3,4-tetrahydro-naphthalen-2-ylmethyl)-2-aza-bicyclo[3.3.1]non-5-yl]-phenyl}-amide;
N-{3-[2-(1-hydroxy-3-phenyl-cyclobutylmethyl)-2-aza-bicyclo[3.3.1]non-5-yl]-phenyl}-methanesulfonamide; and
N-{3-[2-(2-hydroxy-1,2,3,4-tetrahydro-naphthalen-2-ylmethyl)-2-aza-bicyclo[3.3.1]non-5-yl]-phenyl}-methanesulfonamide.

Preferably, the opioid receptor antagonist is selected from:
2-methoxy-ethanesulfonic acid {3-[6-ethyl-3-(2-hydroxy-1,2,3,4-tetrahydro-naphthalen-2-ylmethyl )-3-aza-bicyclo[3.1.0]hex-6-yl]-phenyl}-amide;
N-[3-(6-ethyl-3-indan-2-ylmethyl-3-aza-bicyclo[3.1.0]hex-6-yl)-phenyl]-methanesulfonamide;
2-methoxy-ethanesulfonic acid [3-(6-ethyl-3-indan-2-ylmethyl-3-aza-bicyclo[3.1.0]hex-6-yl)-phenyl]-amide;
N-{3-[6-ethyl-3-(2-hydroxy-indan-2-ylmethyl )-3-aza-bicyclo[3.1.0]hex-6-yl]-phenyl}-methanesulfonamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
N-(3-{6-ethyl-3-[3-(1-hydroxy-cyclohexyl )-propyl]-3-aza-bicyclo[3.1.0]hex-6-yl}-phenyl)-methanesulfonamide;
3-{3-[3-(1-hydroxy-cyclohexyl)-propyl]-8-methoxy-3-aza-bicyclo[3.2.1]oct-8-yl}-benzamide;
2-methoxy-ethanesulfonic acid {3-[6-ethyl-3-(2-hydroxy-indan-2-ylmethyl)-3-aza-bicyclo[3.1.0]hex-6-yl]-phenyl}-amide;
3-[6-ethyl-3-(2-hydroxy-indan-2-ylmethyl)-3-aza-bicyclo[3.1.0]hex-6-yl]-benzamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
3-[6-ethyl-3-(2-hydroxy-1,2,3,4-tetrahydro-naphthalen-2-ylmethyl)-3-aza-bicyclo[3.1.0]hex-6-yl]-benzamide;
N-{3-[2-(2-hydroxy-indan-2-ylmethyl)-2-aza-bicyclo[3.3.1]non-5-yl]-phenyl}-methanesulfonamide;
2-methoxy-ethanesulfonic acid {3-[3-(2-hydroxy-indan-2-ylmethyl)-8-methoxy-3-aza-bicyclo[3.2.1]oct-8-yl]-phenyl}-amide;
3-{6-ethyl-3-[3-(1-hydroxy-cyclohexyl)-propyl]-3-aza-bicyclo[3.1.0]hex-6-yl}-benzamide;
3-[6-ethyl-3-(1-hydroxy-3-phenyl-cyclobutylmethyl)-3-aza-bicyclo[3.1.0]hex-6-yl]-benzamide;
2-methoxy-ethanesulfonic acid (3-{6-ethyl-3-[3-(1-hydroxy-cyclohexyl)-propyl]-3-aza-bicyclo[3.1.0]hex-6-yl}-phenyl)-amide;
3-{1-[3-(1-hydroxy-cyclohexyl)-propyl]-3,4-dimethyl-piperidin-4-yl}-benzamide;
3-(1-indan-2-ylmethyl-3,4-dimethyl-piperidin-4-yl)-benzamide;
N-{3-[3-(2-hydroxy-indan-2-ylmethyl)-8-methoxy-3-aza-bicyclo[3.2.1]oct-8-yl]-phenyl}-methanesulfonamide;
3-[-(1-hydroxy-3-phenyl-cyclobutylmethyl)-3,4-dimethyl-piperidin-4-yl]-benzamide;
3-(6-hthyl-3-indan-2-ylmethyl-3-aza-bicyclo[3.1.0]hex-6-yl)-benzamide;
N-(3-{3-[3-(1-hydroxy-cyclohexyl)-propyl]-8-methoxy-3-aza-bicyclo[3.2.1]oct-8-yl}-phenyl)-methanesulfonamide;
3-[1 -(2-hydroxy-indan-2-ylmethyl)-3,4-dimethyl-piperidin-4-yl]-benzamide;
N-(3-{2-[3-(1-hydroxy-cyclohexyl)-propyl]-2-aza-bicyclo[3.3.1]non-5-yl}-phenyl)-methanesulfonamide;
3-[3-(1-hydroxy-3-phenyl-cyclobutylmethyl)-8-methoxy-3-aza-bicyclo[3.2.1]oct-8-yl]-benzamide;
3-[3-(2-hydroxy-indan-2-yl methyl)-8-methoxy-3-aza-bicyclo[3.2.1]oct-8-yl]-benzamide;
3-[2-(2-hydroxy-indan-2-ylmethyl)-2-aza-bicyclo[3.3.1]non-5-yl]-benzamide;
3-{2-[3-(1-hydroxy-cyclohexyl)-propyl]-2-aza-bicyclo[3.3.1]non-5-yl}-benzamide;
3-[2-(1-hydroxy-3-phenyl-cyclobutylmethyl)-2-aza-bicyclo[3.3.1]non-5-yl]-benzamide;
3-[2-(2-hydroxy-1,2,3,4-tetrahydro-naphthalen-2-ylmethyl)-2-aza-bicyclo[3.3.1]non-5-yl]-benzamide;
2-methoxy-ethanesulfonic acid {3-[2-(2-hydroxy-indan-2-ylmethyl)-2-aza-bicyclo[3.3.1]non-5-yl]-phenyl}-amide;
2-methoxy-ethanesulfonic acid (3-{2-[3-(1-hydroxy-cyclohexyl)-propyl]-2-aza-bicyclo[3.3.1]non-5-yl}-phenyl)-amide;
2-methoxy-ethanesulfonic acid {3-[2-(1-hydroxy-3-phenyl-cyclobutylmethyl)-2-aza-bicyclo[3.3.1]non-5-yl]-phenyl}-amide;
2-methoxy-ethanesulfonic acid {3-[2-(2-hydroxy-1,2,3,4-tetrahydro-naphthalen-2-ylmethyl)-2-aza-bicyclo[3.3.1]non-5-yl]-phenyl}-amide;
N-{3-[2-(1-hydroxy-3-phenyl-cyclobutylmethyl)-2-aza-bicyclo[3.3.1]non-5-yl]-phenyl}-methanesulfonamide; and
N-{3-[2-(2-hydroxy-1,2,3,4-tetrahydro-naphthalen-2-ylmethyl)-2-aza-bicyclo[3.3.1]non-5-yl]-phenyl}-methanesulfonamide.

The present invention also relates to a method of treating alcohol or cocaine dependence or addiction, tobacco dependence or addiction, reducing alcohol withdrawal symptoms or aiding in the cessation or lessening of alcohol use or substance abuse or behavioral dependencies, including gambling, comprising:
(a) an opioid receptor antagonist or a pharmaceutically acceptable salt thereof; and
(b) a CB-1 receptor antagonist or pharmaceutically acceptable salt thereof; and
(c) a pharmaceutically acceptable carrier,
wherein the active agents (a) and (b) above are present in amounts that render the composition effective in treating alcohol or cocaine dependence or addiction, tobacco dependence or addiction, reducing alcohol withdrawal symptoms or aiding in the cessation or lessening of alcohol use or substance abuse or behavioral dependencies.

The CB-1 receptor antagonist and the opioid receptor antagonist present in amounts that render the composition effective in the treatment of alcohol, cocaine or nicotine addiction, alcohol withdrawal symptoms, substance abuse or other behavioral dependencies. In a more specific embodiment of the invention, the CB-1 receptor antagonists are listed herein above.

In another more specific embodiment of this invention the opioid receptor antagonist is selected from:
2-methoxy-ethanesulfonic acid {3-[6-ethyl-3-(2-hydroxy-1,2,3,4-tetrahydro-naphthalen-2-ylmethyl)-3-aza-bicyclo[3.1.0]hex-6-yl]-phenyl}-amide;
N-[3-(6-ethyl-3-indan-2-ylmethyl-3-aza-bicyclo[3.1.0]hex-6-yl)-phenyl]-methanesulfonamide;
2-methoxy-ethanesulfonic acid [3-(6-ethyl-3-indan-2-ylmethyl-3-aza-bicyclo[3.1.0]hex-6-yl)-phenyl]-amide;
N-{3-[6-ethyl-3-(2-hydroxy-indan-2-ylmethyl)-3-aza-bicyclo[3.1.0]hex-6-yl]-phenyl}-methanesulfonamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
N-(3-{6-ethyl-3-[3-(1-hydroxy-cyclohexyl)-propyl]-3-aza-bicyclo[3.1.0]hex-6-yl}-phenyl)-methanesulfonamide;
3-{3-[3-(1-hydroxy-cyclohexyl)-propyl]-8-methoxy-3-aza-bicyclo[3.2.1]oct-8-yl}-benzamide;
2-methoxy-ethanesulfonic acid {3-[6-ethyl-3-(2-hydroxy-indan-2-ylmethyl)-3-aza-bicyclo[3.1.0]hex-6-yl]-phenyl}-amide;
3-[6-ethyl-3-(2-hydroxy-indan-2-ylmethyl)-3-aza-bicyclo[3.1.0]hex-6-yl]-benzamide;
N-{3-[6-ethyl-3-(2-hydroxy-1,2,3,4-tetrahydro-naphthalen-2-ylmethyl)-3-aza-bicyclo[3.1.09 hex-6-yl]-phenyl}-methanesulfonamide;
3-[6-ethyl-3-(2-hydroxy-1,2,3,4-tetrahydro-naphthalen-2-ylmethyl )-3-aza-bicyclo[3.1.0]hex-6-yl]-benzamide;
N-{3-[2-(2-hydroxy-indan-2-ylmethyl)-2-aza-bicyclo[3.3.1]non-5-yl]-phenyl}-methanesulfonamide;
2-methoxy-ethanesulfonic acid {3-[3-(2-hydroxy-indan-2-ylmethyl)-8-methoxy-3-aza-bicyclo[3.2.1]oct-8-yl]-phenyl}-amide;
3-{6-ethyl-3-[3-(1-hydroxy-cyclohexyl )-propyl]-3-aza-bicyclo[3.1.0]hex-6-yl}-benzamide;
3-[6-ethyl-3-(1-hydroxy-3-phenyl-cyclobutylmethyl)-3-aza-bicyclo[3.1.0]hex-6-yl]-benzamide;
2-methoxy-ethanesulfonic acid (3-{6-ethyl-3-[3-(1-hydroxy-cyclohexyl)-propyl]-3-aza-bicyclo[3.1.0]hex-6-yl}-phenyl)-amide;
3-{1 -[3-(1-hydroxy-cyclohexyl)-propyl]-3,4-dimethyl-piperidin-4-yl}-benzamide;
3-(1-indan-2-ylmethyl-3,4-dimethyl-piperidin-4-yl)-benzamide;
N-{3-[3-(2-hydroxy-indan-2-ylmethyl)-8-methoxy-3-aza-bicyclo[3.2.1]oct-8-yl]-phenyl}-methanesulfonamide;
3-[1-(1-hydroxy-3-phenyl-cyclobutylmethyl)-3,4-dimethyl-piperidin-4-yl]-benzamide;
3-(6-hthyl-3-indan-2-ylmethyl-3-aza-bicyclo[3.1.0]hex-6-yl)-benzamide;
N-(3-{3-[3-(1-hydroxy-cyclohexyl)-propyl]-8-methoxy-3-aza-bicyclo[3.2.1]oct-8-yl}-phenyl)-methanesulfonamide;
3-[1-(2-hydroxy-indan-2-ylmethyl)-3,4-dimethyl-piperidin-4-yl]-benzamide;
N-(3-{2-[3-(1-hydroxy-cyclohexyl)-propyl]-2-aza-bicyclo[3.3.1]non-5-yl}-phenyl)-methanesulfonamide;
3-[3-(1-hydroxy-3-phenyl-cyclobutylmethyl)-8-methoxy-3-aza-bicyclo[3.2.1]oct-8-yl]-benzamide;
3-[3-(2-hydroxy-indan-2-ylmethyl)-8-methoxy-3-aza-bicyclo[3.2.1]oct-8-yl]-benzamide;
3-[2-(2-hydroxy-indan-2-yl methyl)-2-aza-bicyclo[3.3.1]non-5-yl]-benzamide;
3-{2-[3-(1-hydroxy-cyclohexyl)-propyl]-2-aza-bicyclo[3.3.1]non-5-yl}-benzamide;
3-[2-(1-hydroxy-3-phenyl-cyclobutylmethyl)-2-aza-bicyclo[3.3.1]non-5-yl]-benzamide;
3-[2-(2-hydroxy-1,2,3,4-tetrahydro-naphthalen-2-ylmethyl)-2-aza-bicyclo[3.3.1]non-5-yl]-benzamide;
2-methoxy-ethanesulfonic acid {3-[2-(2-hydroxy-indan-2-ylmethyl)-2-aza-bicyclo[3.3.1]non-5-yl]-phenyl}-amide;
2-methoxy-ethanesulfonic acid (3-{2-[3-(1-hydroxy-cyclohexyl)-propyl]-2-aza-bicyclo[3.3.1]non-5-yl}-phenyl)-amide;
2-methoxy-ethanesulfonic acid {3-[2-(1-hydroxy-3-phenyl-cyclobutylmethyl)-2-aza-bicyclo[3.3.1]non-5-yl]-phenyl}-amide;
2-methoxy-ethanesulfonic acid {3-[2-(2-hydroxy-1,2,3,4-tetrahydro-naphthalen-2-ylmethyl)-2-aza-bicyclo[3.3.1]non-5-yl]-phenyl}-amide;
N-{3-[2-(1-hydroxy-3-phenyl-cyclobutylmethyl)-2-aza-bicyclo[3.3.1]non-5-yl]-phenyl}-methanesulfonamide; and
N-{3-[2-(2-hydroxy-1,2,3,4-tetrahydro-naphthalen-2-ylmethyl)-2-aza-bicyclo[3.3.1]non-5-yl]-phenyl}-methanesulfonamide.

Preferably, the opioid receptor antagonist is selected from:
2-methoxy-ethanesulfonic acid {3-[6-ethyl-3-(2-hydroxy-1,2,3,4-tetrahydro-naphthalen-2-ylmethyl)-3-aza-bicyclo[3.1.0]hex-6-yl]-phenyl}-amide;
N-[3-(6-ethyl-3-indan-2-ylmethyl-3-aza-bicyclo[3.1.0]hex-6-yl)-phenyl]-methanesulfonamide;
2-methoxy-ethanesulfonic acid [3-(6-ethyl-3-indan-2-ylmethyl-3-aza-bicyclo[3.1.0]hex-6-yl)-phenyl]-amide;
N-{3-[6-ethyl-3-(2-hydroxy-indan-2-ylmethyl)-3-aza-bicyclo[3.1.0]hex-6-yl]-phenyl}-methanesulfonamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
N-(3-{6-ethyl-3-[3-(1-hydroxy-cyclohexyl)-propyl]-3-aza-bicyclo[3.1.0]hex-6-yl}-phenyl)-methanesulfonamide;
3-{3-[3-(1-hydroxy-cyclohexyl)-propyl]-8-methoxy-3-aza-bicyclo[3.2.1]oct-8-yl}-benzamide;
2-methoxy-ethanesulfonic acid {3-[6-ethyl-3-(2-hydroxy-indan-2-ylmethyl)-3-aza-bicyclo[3.1.0]hex-6-yl]-phenyl}-amide;
3-[6-ethyl-3-(2-hydroxy-indan-2-ylmethyl)-3-aza-bicyclo[3.1.0]hex-6-yl]-benzamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
3-[6-ethyl-3-(2-hydroxy-1,2,3,4-tetrahydro-naphthalen-2-ylmethyl)-3-aza-bicyclo[3.1.0]hex-6-yl]-benzamide;
N-{3-[2-(2-hydroxy-indan-2-ylmethyl)-2-aza-bicyclo[3.3.1]non-5-yl]-phenyl}-methanesulfonamide;
2-methoxy-ethanesulfonic acid {3-[3-(2-hydroxy-indan-2-ylmethyl)-8-methoxy-3-aza-bicyclo[3.2.1]oct-8-yl]-phenyl}-amide;
3-{6-ethyl-3-[3-(1-hydroxy-cyclohexyl )-propyl]-3-aza-bicyclo[3.1.0]hex-6-yl}-benzamide;
3-[6-ethyl-3-(1-hydroxy-3-phenyl-cyclobutylmethyl)-3-aza-bicyclo[3.1.0]hex-6-yl]-benzamide;
2-methoxy-ethanesulfonic acid (3-{6-ethyl-3-[3-(1-hydroxy-cyclohexyl)-propyl]-3-aza-bicyclo[3.1.0]hex-6-yl}-phenyl)-amide;
3-{1 -[3-(1-hydroxy-cyclohexyl)-propyl]-3,4-dimethyl-piperidin-4-yl}-benzamide;
3-(1-indan-2-ylmethyl-3,4-dimethyl-piperidin-4-yl)-benzamide;
N-{3-[3-(2-hydroxy-indan-2-ylmethyl)-8-methoxy-3-aza-bicyclo[3.2.1]oct-8-yl]-phenyl}-methanesulfonamide;
3-[1-(1-hydroxy-3-phenyl-cyclobutylmethyl)-3,4-dimethyl-piperidin-4-yl]-benzamide;
3-(6-hthyl-3-indan-2-ylmethyl-3-aza-bicyclo[3.1.0]hex-6-yl)-benzamide;
N-(3-{3-[3-(1-hydroxy-cyclohexyl)-propyl]-8-methoxy-3-aza-bicyclo[3.2.1]oct-8-yl}-phenyl)-methanesulfonamide;
3-[1 -(2-hydroxy-indan-2-ylmethyl)-3,4-dimethyl-piperidin-4-yl]-benzamide;
N-(3-{2-[3-(1-hydroxy-cyclohexyl)-propyl]-2-aza-bicyclo[3.3.1]non-5-yl}-phenyl)-methanesulfonamide;
3-[3-(1-hydroxy-3-phenyl-cyclobutylmethyl)-8-methoxy-3-aza-bicyclo[3.2.1]oct-8-yl]-benzamide;
3-[3-(2-hydroxy-indan-2-ylmethyl)-8-methoxy-3-aza-bicyclo[3.2.1]oct-8-yl]-benzamide;
3-[2-(2-hydroxy-indan-2-ylmethyl)-2-aza-bicyclo[3.3.1]non-5-yl]-benzamide;
3-{2-[3-(1-hydroxy-cyclohexyl)-propyl]-2-aza-bicyclo[3.3.1]non-5-yl}-benzamide;
3-[2-(1-hydroxy-3-phenyl-cyclobutylmethyl)-2-aza-bicyclo[3.3.1]non-5-yl]-benzamide;
3-[2-(2-hydroxy-1,2,3,4-tetrahydro-naphthalen-2-ylmethyl)-2-aza-bicyclo[3.3.1]non-5-yl]-benzamide;
2-methoxy-ethanesulfonic acid {3-[2-(2-hydroxy-indan-2-ylmethyl)-2-aza-bicyclo[3.3.1]non-5-yl]-phenyl}-amide;
2-methoxy-ethanesulfonic acid (3-{2-[3-(1-hydroxy-cyclohexyl)-propyl]-2-aza-bicyclo[3.3.1]non-5-yl}-phenyl)-amide;
2-methoxy-ethanesulfonic acid {3-[2-(1-hydroxy-3-phenyl-cyclobutylmethyl)-2-aza-bicyclo[3.3.1]non-5-yl]-phenyl}-amide;
2-methoxy-ethanesulfonic acid {3-[2-(2-hydroxy-1,2,3,4-tetrahydro-naphthalen-2-ylmethyl)-2-aza-bicyclo[3.3.1]non-5-yl]-phenyl}-amide;
N-{3-[2-(1-hydroxy-3-phenyl-cyclobutylmethyl)-2-aza-bicyclo[3.3.1]non-5-yl]-phenyl}-methanesulfonamide; and
N-{3-[2-(2-hydroxy-1,2,3,4-tetrahydro-naphthalen-2-ylmethyl)-2-aza-bicyclo[3.3.1]non-5-yl]-phenyl}-methanesulfonamide.

The term “treating” as used herein, refers to reversing, alleviating, inhibiting or slowing the progress of, or preventing the disorder or condition to which such term applies, or one or more symptoms of such disorder or condition. The term “treatment”, as used herein, refers to the act of treating, as “treating” is defined immediately above.

The term “substance abuse”, as used herein, for example in “drug addiction” and “alcohol addiction”, unless otherwise indicated, refers to a maladaptive use of a substance, which may be either with physiological dependence or without. The term “substance abuse” thus includes both substance abuse (e.g. nicotine, alcohol, amphetamine, cocaine or an opioid, for example morphine, opium, or heroine, abuse) and substance dependence (e.g. nicotine, alcohol, amphetamine, cocaine or an opioid, for example morphine, opium, or heroine dependence). The maladaptive pattern of substance use may manifest itself in recurrent and significant adverse consequences related to the repeated use of the substance. The recurrent substance use may result in a failure to fulfill major role obligations at work, school, or home. The maladaptive use of a substance may involve continued use of the substance despite persistent or recurrent social or interpersonal problems caused or exacerbated by the effects of the substance (e.g., arguments with spouse, physical fights). The maladaptive pattern of substance use may involve clinically significant impairment or distress, for example manifested by tolerance for the substance, withdrawal symptoms, unsuccessful efforts to cut down or control the substance use, and/or taking larger amounts of the substance and/or taking amounts of the substance over a longer period than was intended. Substances to which an addiction may be formed include, but are not limited to, the drugs recited above (including nicotine, alcohol), as well as others, for example benzodiazepines such as Valium®.

Behavioral dependencies as used here means enduring or persistent patterns of behavior which deviates markedly from the expectations of an individual's culture, is pervasive and inflexible, is stable over time, and leads to distress or impairment, and can include either Axis I or Axis II diagnoses (1994; DSM-IV, American Psychiatric Association). Such diagnoses may include, but are not limited to, substance abuse (nicotine, alcohol, narcotics, inhalants), gambling, eating disorders, and impulse control disorders.

The chemist of ordinary skill will recognize that certain compounds of this invention will contain one or more atoms which may be in a particular stereochemical or geometric configuration, giving rise to stereoisomers and configurational isomers. All such isomers and mixture thereof are included in this invention. Hydrates of the compounds of this invention are also included.

The chemist of ordinary skill will recognize that certain combinations of heteroatom-containing substituent listed in this invention define compounds which will be less stable under physiological conditions (e.g., those containing acetal or animal linkages). According, such compounds are less preferred.

### DETAILED DESCRIPTION OF THE INVENTION

In combination with the opioid receptor antagonist, the invention includes a CB-1 receptor antagonist and a pharmaceutically acceptable salt thereof.

The particular opioid receptor ligands listed above, which can be employed in the methods and pharmaceutical compositions of this invention, can be made by processes known in the chemical arts, for example by the methods described in WO 03/035,622 published May 1, 2003 which is U.S. Ser. No. 10/278,142 and 60/462,651 filed Apr. 14, 2003 and 60/462,629 filed Apr. 14, 2003 and 60/462,605 filed Apr. 14, 2003 which are incorporated by reference their entireties.

Some of the preparation methods useful for making the compounds of this invention may require protection of remote functionality (i.e., primary amine, secondary amine, carboxyl). The need for such protection will vary depending on the nature of the remote functionality and the conditions of the preparation methods. The need for such protection is readily determined by one skilled in the art, and is described in examples carefully described in the above cited applications. The starting materials and reagents for the opioid receptor antagonist employed in this invention are also readily available or can be easily synthesized by those skilled in the art using conventional methods of organic synthesis. Some of the compounds used herein are related to, or are derived from compounds found in nature and accordingly many such compounds are commercially available or are reported in the literature or are easily prepared from other commonly available substances by methods which are reported in the literature.

Some of the opioid receptor antagonist compounds employed in this invention are ionizable at physiological conditions. Thus, for example some of the compounds of this invention are acidic and they form a salt with a pharmaceutically acceptable cation. All such salts are within the scope of this invention and they can be prepared by conventional methods. For example, they can be prepared simply by contacting the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate.

In addition, some of the opioid receptor antagonist employed in this invention are basic, and they form a salt with a pharmaceutically acceptable anion. All such salts are within the scope of this invention and they can be prepared by conventional methods. For example, they can be prepared simply by contacting the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate.

In addition, when the opioid receptor antagonists employed in this invention form hydrates or solvates they are also within the scope of the invention.

Some of the compounds of this invention are chiral, and as such are subject to preparation via chiral synthetic routes, or separable by conventional resolution or chromatographic means. All optical forms of the compounds of this invention are within the scope of the invention.

The utility of the opioid receptor antagonists employed in the present invention as medicinal agents in the treatment of alcohol dependence (such as substance dependence or addiction) in mammals (e.g. humans) is demonstrated by the activity of the compounds of this invention in conventional assays and, in particular the assays described below. Such assays also provide a means whereby the activities of the compounds of this invention can be compared between themselves and with the activities of other known compounds. The results of these comparisons are useful for determining dosage levels in mammals, including humans, for the treatment of such diseases.

### BIOLOGICAL ASSAYS

### Procedures

Biological Activity

Compounds of the subject invention have been found to display activity in opioid receptor binding assays selective for the mu, kappa and delta opioid receptors. Assays for mu, kappa and delta opioid receptor binding can be performed according to the following procedure:

Affinity of a compound for the delta opioid receptor can be assessed using binding of the delta opioid receptor ligand [³H]-naltrindole to NG108-15 neuroblastoma-glioma cells according to modification of the protocol described in Law et al. (Law, P. Y., Koehler, J. E. and Loh, H. H., “Comparison of Opioid Inhibition of Adenylate Cyclase Activity in Neuroblastoma N18TG2 and Neuroblastoma X Glioma NG108-15 Hybrid Cell Lines”, Molecular Pharmacology, 21: 483-491 (1982)). Law et al. is incorporated herein in its entirety by reference. Affinity of a compound for the kappa opioid receptor can be assessed using binding of [³H]-bremazocine to kappa receptors as described in Robson, L. E., et al., “Opioid Binding Sites of the Kappa-type in Guinea-pig Cerebellum”, Neuroscience (Oxford), 12(2): 621-627 (1984). Robson et al. is incorporated herein it its entirety by reference. For assessment of a compound for mu opioid receptor activity, the mu receptor ligand [³H]-DAMGO (Perkin Elmer Life Sciences, Boston, Mass.; specific activity 55Ci/mmol, 1.5 nM) is used with rat forebrain tissue. Briefly, the binding is initiated with the addition of a crude membrane preparation of rat forebrain tissue to 96-well polypropylene plates containing the radioligand [³H]-DAMGO and test compound, and are incubated for about 90 minutes at about 25° C. The assay is terminated by rapid filtration with 50 mM Tris HCl pH 7.4 onto Wallac Filtermat B and counted on a Betaplate reader (Wallac).

The data generated can be analyzed using IC₅₀ analysis software in Graphpad Prism. Ki values can be calculated using Graphpad Prism according to the following formula:
Ki=IC₅₀/1+[³H ligand]/K_{D}
where IC₅₀ is the concentration at which 50% of the ³H ligand is displaced by the test compound and K_{D} is the dissociation constant for the ³H ligand at the receptor site.

The Ki values of certain compounds I of the Examples, as described, infra, in a mu opioid receptor binding assay to brain tissue such as that described above, were determined. All of the compounds tested in this manner were all found to have Ki values of about 800 nM or less for the mu opioid receptor.

The inhibition (%) of [³H]-DAMGO binding by certain compounds of the Examples, as described, infra, in a mu opioid receptor binding assay to brain tissue such as that described above, were determined. Most of the compounds tested at 100 nM were found to inhibit [³H]-DAMGO binding at the mu opioid receptor in a range of 10-100%.

Pharmacological Testing of CB-1 Receptor Antagonists

The utility of the compounds of the present invention in the practice of the instant invention can be evidenced by activity in at least one of the protocols described hereinbelow. The following acronyms are used in the protocols described below.
BSA—bovine serum albumin
DMSO—dimethylsulfoxide
EDTA—ethylenediamine tetracetic acid
PBS—phosphate-buffered saline
EGTA—ethylene glycol-bis(β-aminoethyl ether) N,N,N′,N′-tetraacetic acid
GDP—guanosine diphosphate
sc—subcutaneous
po—orally
ip—intraperitoneal
icv—intra cerebro ventricular
iv—intravenous

³H]SR141716A—radiolabeled N-(piperidin-1-yl)-5-(4-chlorophenyl)-1-(2,4-dichlorophenyl)-4-methyl-1H-pyrazole-3-carboxamide hydrochloride available from Amersham Biosciences, Piscataway, N.J.

[³H]CP-55940—radiolabled 5-(1,1-dimethylheptyl)-2-[5-hydroxy-2-(3-hydroxypropyl)-cyclohexyl]-phenol available from NEN Life Science Products, Boston, Mass.

AM251—N-(piperidin-1-yl)-1-(2,4-dichlorophenyl)-5-(4-iodophenyl)-4-methyl-1H-pyrazole-3-carboxamide available from Tocris™, Ellisville, Mo.

### In Vitro Biological Assays

Bioassay systems for determining the CB-1 and CB-2 binding properties and pharmacological activity of cannabinoid receptor ligands are described by Roger G. Pertwee in “Pharmacology of Cannabinoid Receptor Ligands” Current Medicinal Chemistry, 6, 635-664 (1999) and in WO 92/02640 (U.S. application Ser. No. 07/564,075 filed Aug. 8, 1990, incorporated herein by reference).

The following assays were designed to detect compounds that inhibit the binding of [³H] SR141716A (selective radiolabeled CB-1 ligand) and [³H] 5-(1,1-dimethylheptyl)-2-[5-hydroxy-2-(3-hydroxypropyl)-cyclohexyl]-phenol ([³H]CP-55940; radiolabeled CB-1 /CB-2 ligand) to their respective receptors.

### Rat CB-1 Receptor Binding Protocol

PelFreeze brains (available from Pel Freeze Biologicals, Rogers, Ark.) were cut up and placed in tissue preparation buffer (5 mM Tris HCl, pH=7.4 and 2 mM EDTA), polytroned at high speed and kept on ice for 15 minutes. The homogenate was then spun at 1,000× g for 5 minutes at 4° C. The supernatant was recovered and centrifuged at 100,000× G for 1 hour at 4° C. The pellet was then re-suspended in 25 ml of TME (25 nM Tris, pH=7.4, 5 mM MgCl₂, and 1 mM EDTA) per brain used. A protein assay was performed and 200 μl of tissue totaling 20 μg was added to the assay.

The test compounds were diluted in drug buffer (0.5% BSA, 10% DMSO and TME) and then 25 μl were added to a deep well polypropylene plate. [³H] SR141716A was diluted in a ligand buffer (0.5% BSA plus TME) and 25 μl were added to the plate. A BCA protein assay was used to determine the appropriate tissue concentration and then 200 μl of rat brain tissue at the appropriate concentration was added to the plate. The plates were covered and placed in an incubator at 20° C. for 60 minutes. At the end of the incubation period 250 μl of stop buffer (5% BSA plus TME) was added to the reaction plate. The plates were then harvested by Skatron onto GF/B filtermats presoaked in BSA (5 mg/ml) plus TME. Each filter was washed twice. The filters were dried overnight. In the morning the filters were counted on a Wallac Betaplate™ counter (available from PerkinElmer Life Sciences™, Boston, Mass.).

### Human CB-1 Receptor Binding Protocol

Human embryonic kidney 293 (HEK 293) cells transfected with the CB-1 receptor cDNA (obtained from Dr. Debra Kendall, University of Connecticut) were harvested in homogenization buffer (10 mM EDTA, 10 mM EGTA, 10 mM Na Bicarbonate, protease inhibitors; pH=7.4), and homogenized with a Dounce Homogenizer. The homogenate was then spun at 1,000× g for 5 minutes at 4° C. The supernatant was recovered and centrifuged at 25,000× G for 20 minutes at 4° C. The pellet was then re-suspended in 10 ml of homogenization buffer and re-spun at 25,000× G for 20 minutes at 4° C. The final pellet was re-suspended in 1 ml of TME (25 mM Tris buffer (pH=7.4) containing 5 mM MgCl₂ and 1 mM EDTA). A protein assay was performed and 200 μl of tissue totaling 20 μg was added to the assay.

The test compounds were diluted in drug buffer (0.5% BSA, 10% DMSO and TME) and then 25 μl were added to a deep well polypropylene plate. [³H]SR141716A was diluted in a ligand buffer (0.5% BSA plus TME) and 25 μl were added to the plate. The plates were covered and placed in an incubator at 30° C. for 60 minutes. At the end of the incubation period 250 μl of stop buffer (5% BSA plus TME) was added to the reaction plate. The plates were then harvested by Skatron onto GF/B filtermats presoaked in BSA (5 mg/ml) plus TME. Each filter was washed twice. The filters were dried overnight. In the morning the filters were counted on a Wallac Betaplate™ counter (available from PerkinElmer Life Sciences™, Boston, Mass.).

### CB-2 Receptor Binding Protocol

Chinese hamster ovary-K1 (CHO-K1) cells transfected with CB-2 cDNA (obtained from Dr. Debra Kendall, University of Connecticut) were harvested in tissue preparation buffer (5 mM Tris-HCl buffer (pH=7.4) containing 2 mM EDTA), polytroned at high speed and kept on ice for 15 minutes. The homogenate was then spun at 1,000× g for 5 minutes at 4° C. The supernatant was recovered and centrifuged at 100,000× G for 1 hour at 4° C. The pellet was then re-suspended in 25 ml of TME (25 mM Tris buffer (pH=7.4) containing 5 mM MgCl₂ and 1 mM EDTA) per brain used. A protein assay was performed and 200 μl of tissue totaling 10 μg was added to the assay.

The test compounds were diluted in drug buffer (0.5% BSA, 10% DMSO, and 80.5% TME) and then 25 μl were added to the deep well polypropylene plate. [³H] CP-55940 was diluted a ligand buffer (0.5% BSA and 99.5% TME) and then 25 μl were added to each well at a concentration of 1 nM. A BCA protein assay was used to determine the appropriate tissue concentration and 200 μl of the tissue at the appropriate concentration was added to the plate. The plates were covered and placed in an incubator at 30° C. for 60 minutes. At the end of the incubation period 250 μl of stop buffer (5% BSA plus TME) was added to the reaction plate. The plates were then harvested by Skatron format onto GF/B filtermats presoaked in BSA (5 mg/ml) plus TME. Each filter was washed twice. The filters were dried overnight. The filters were then counted on the Wallac Betaplate™ counter.

### CB-1 GTPγ [³⁵S] Binding Assay

Membranes were prepared from CHO-K1 cells stably transfected with the human CB-1 receptor cDNA. Membranes were prepared from cells as described by Bass et al, in “Identification and characterization of novel somatostatin antagonists,” Molecular Pharmacology, 50, 709-715 (1996). GTPγ [³⁵S] binding assays were performed in a 96 well FlashPlate™ format in duplicate using 100 μM GTPγ[³⁵S] and 10 μg membrane per well in assay buffer composed of 50 mM Tris HCl, pH 7.4, 3 mM MgCl₂, pH 7.4, 10 mM MgCl₂, 20 mM EGTA, 100 mM NaCl, 30 μM GDP, 0.1% bovine serum albumin and the following protease inhibitors: 100 μg/ml bacitracin, 100 μg/ml benzamidine, 5 μg/ml aprotinin, 5 μg/ml leupeptin. The assay mix was then incubated with increasing concentrations of antagonist (10⁻¹⁰ M to 10⁻⁵ M) for 10 minutes and challenged with the cannabinoid agonist CP-55940 (10 μM). Assays were performed at 30° C. for one hour. The FlashPlates™ were then centrifuged at 2000× g for 10 minutes. Stimulation of GTPγ[³⁵S] binding was then quantified using a Wallac Microbeta.EC₅₀ calculations done using Prism™ by Graphpad.

Inverse agonism was measured in the absense of agonist.

### CB-1 FLIPR-Based Functional Assay Protocol

CHO-K1 cells co-transfected with the human CB-1 receptor cDNA (obtained from Dr. Debra Kendall, University of Connecticut) and the promiscuous G-protein G16 were used for this assay. Cells were plated 48 hours in advance at 12500 cells per well on collagen coated 384 well black clear assay plates. Cells were incubated for one hour with 4 μM Fluo-4 AM (Molecular Probes) in DMEM (Gibco) containing 2.5 mM probenicid and pluronic acid (0.04%). The plates were then washed 3 times with HEPES-buffered saline (containing probenicid; 2.5 mM) to remove excess dye. After 20 min the plates were added to the FLIPR individually and fluorescence levels was continuously monitored over an 80 s period. Compound additions were made simultaneously to all 384 wells after 20 s of baseline. Assays were performed in triplicate and 6 point concentration-response curves generated. Antagonist compounds were subsequently challenged with 3 μM WIN 55,212-2 (agonist). Data were analyzed using Graph Pad Prism.

### Detection of Inverse Agonists

The following cyclic-AMP assay protocol using intact cells was used to determine inverse agonist activity.

Cells were plated into a 96-well plate at a plating density of 10,000-14,000 cells per well at a concentration of 100 μl per well. The plates were incubated for 24 hours in a 37° C. incubator. The media was removed and media lacking serum (100 μl) was added. The plates were then incubated for 18 hours at 37° C.

Serum free medium containing 1 mM IBMX was added to each well followed by 10 μl of test compound (1:10 stock solution (25 mM compound in DMSO) into 50% DMSO/PBS) diluted 10× in PBS with 0.1% BSA. After incubating for 20 minutes at 37° C., 2 μM of Forskolin was added and then incubated for an additional 20 minutes at 37° C. The media was removed, 100 μl of 0.01N HCl was added and then incubated for 20 minutes at room temperature. Cell lysate (75 μl) along with 25 μl of assay buffer (supplied in FlashPlate™ cAMP assay kit available from NEN Life Science Products Boston, Mass.) into a Flashplate. cAMP standards and cAMP tracer were added following the kit's protocol. The flashplate was then incubated for 18 hours at 4° C. The content of the wells were aspirated and counted in a Scintillation counter.

### In Vivo Biological Assays

Cannabinoid agoinists such as Δ⁹-tetrahydrocannabinol (Δ⁹-THC) and CP-55940 have been shown to affect four characteristic behaviors in mice, collectively known as the Tetrad. For a description of these behaviors see: Smith, P. B., et al. in “The pharmacological activity of anandamide, a putative endogenous cannabinoid, in mice.” J. Pharmacol. Exp. Ther., 270(1), 219-227 (1994) and Wiley, J., et al. in “Discriminative stimulus effects of anandamide in rats,” Eur. J. Pharmacol., 276(1-2), 49-54 (1995). Reversal of these activities in the Locomotor Activity, Catalepsy, Hypothermia, and Hot Plate assays described below provides a screen for in vivo activity of CB-1 antagonists.

All data is presented as % reversal from agonist alone using the following formula: (CP/agonist−vehicle/agonist)/(vehicle/vehicle−vehicle/agonist). Negative numbers indicate a potentiation of the agonist activity or non-antagonist activity. Positive numbers indicate a reversal of activity for that particular test.

### Locomotor Activity

Male ICR mice (n=6) (17-19 g, Charles River Laboratories, Inc., Wilmington, Mass.) were pre-treated with test compound (sc, po, ip, or icv). Fifteen minutes later, the mice were challenged with CP-55940 (sc). Twenty-five minutes after the agonist injection, the mice were placed in clear acrylic cages (431.8 cm×20.9 cm×20.3 cm) containing clean wood shavings. The subjects were allowed to explore surroundings for a total of about 5 minutes and the activity was recorded by infrared motion detectors (available from Coulbourn Instruments™, Allentown, Pa.) that were placed on top of the cages. The data was computer collected and expressed as “movement units.”

### Catalepsy

Male ICR mice (n=6)(17-19 g upon arrival) were pre-treated with test compound (sc, po, ip or icv). Fifteen minutes later, the mice were challenged with CP-55940 (sc). Ninety minutes post injection, the mice were placed on a 6.5 cm steel ring attached to a ring stand at a height of about 12 inches. The ring was mounted in a horizontal orientation and the mouse was suspended in the gap of the ring with fore- and hind-paws gripping the perimeter. The duration that the mouse remained completely motionless (except for respiratory movements) was recorded over a 3-minute period.

The data were presented as a percent immobility rating. The rating was calculated by dividing the number of seconds the mouse remains motionless by the total time of the observation period and multiplying the result by 100. A percent reversal from the agonist was then calculated.

### Hypothermia

Male ICR mice (n=5) (17-19 g upon arrival) were pretreated with test compounds (sc, po, ip or icv). Fifteen minutes later, mice were challenged with the cannabinoid agonist CP-55940 (sc). Sixty-five minutes post agonist injection, rectal body temperatures were taken. This was done by inserting a small thermostat probe approximately 2-2.5 cm into the rectum. Temperatures were recorded to the nearest tenth of a degree

### Hot Plate

Male ICR mice (n=7) (17-19 g upon arrival) are pre-treated with test compounds (sc, po, ip or iv). Fifteen minutes later, mice were challenged with a cannabinoid agonist CP-55940 (sc). Forty-five minutes later, each mouse was tested for reversal of analgesia using a standard hot plate meter (Columbus Instruments). The hot plate was 10″×10″×0.75″ with a surrounding clear acrylic wall. Latency to kick, lick or flick hindpaw or jump from the platform was recorded to the nearest tenth of a second. The timer was experimenter activated and each test had a 40 second cut off. Data were presented as a percent reversal of the agonist induced analgesia.

Administration of the compositions of this invention can be via any method which delivers a compound of this invention systemically and/or locally. These methods include oral routes and transdermal routes, etc. Generally, the compounds of this invention are administered orally, but parenteral administration may be utilized (e.g., intravenous, intramuscular, subcutaneous or intramedullary). The two different compounds of this invention can be co-administered simultaneously or sequentially in any order, or a single pharmaceutical composition comprising an opioid receptor as described above and a CB-1 receptor antagonist as described above in a pharmaceutically acceptable carrier can be administered.

The amount and timing of compounds administered will, of course, be based on the judgement of the prescribing physician. Thus, because of patient-to-patient variability, the dosages given below are a guideline and the physician may titrate doses of the agent to achieve the activity that the physician considers appropriate for the individual patient. In considering the degree of activity desired, the physician must balance a variety of factors such as cognitive function, age of the patient, presence of preexisting disease, as well as presence of other diseases (e.g., cardiovascular). The following paragraphs provide preferred dosage ranges for the various components of this invention (based on average human weight of 70 kg).

In general, an effective dosage for the opioid receptor compound or a pharmaceutically acceptable salt thereof can be administered orally, transdermally (e.g., through the use of a patch), parenterally (e.g. intravenously), rectally, topically, or by inhalation. In general, the daily dosage for treating a disorder or condition as described herein will be about from about 0.01 to about 100 mg per kg, preferably from about 0.1 to about 10 mg per kg, of the body weight of the animal to be treated. As an example, a compound or a pharmaceutically acceptable salt thereof, can be administered for treatment to an adult human of average weight (about 70 kg) in a dose ranging from about 0.1 mg up to about 10 g per day, preferably from about 1 mg to about 1 g per day, in single or divided (i.e., multiple) portions. Variations based on the aforementioned dosage ranges may be made by a physician of ordinary skill taking into account known considerations such as the weight, age, and condition of the animal being treated, the severity of the affliction, and the particular route of administration chosen.

In general, an effective dosage for the CB-1 receptor antagonists when used in the combination compositions and methods of this invention is in the range of 0.001 to 200 mg/kg/day, preferably 0.005 to 10.0 mg/kg/day.

The compositions of the present invention are generally administered in the form of a pharmaceutical composition comprising at least one of the compounds of this invention together with a pharmaceutically acceptable vehicle or diluent. Thus, the compounds of this invention can be administered individually or together in any conventional oral, parenteral or transdermal dosage form.

For oral administration a pharmaceutical composition can take the form of solutions, suspensions, tablets, pills, capsules, powders, and the like. Tablets containing various excipient such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g.,topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples, see Remington's Pharmaceutical Sciences, Mack Publishing Company, Easter, Pa., 15th Edition (1975).

Pharmaceutical compositions according to the invention may contain 0.1%-95% of the compound(s) of this invention, preferably 1%-70%. In any event, the composition or formulation to be administered will contain a quantity of a compound(s) according to the invention in an amount effective to treat the dependence of the subject being treated.

1. A pharmaceutical composition for treating alcohol or cocaine dependence or addiction, tobacco dependence or addiction, reducing alcohol withdrawal symptoms or aiding in the cessation or lessening of alcohol use or substance abuse or behavioral dependencies, including gambling, comprising:
(a) an opioid receptor antagonist or a pharmaceutically acceptable salt thereof;
(b) a CB-1 receptor antagonist or pharmaceutically acceptable salt thereof; and
(c) a pharmaceutically acceptable carrier;
wherein the active agents “a” and “b” above are present in amounts that render the composition effective in treating alcohol or cocaine dependence or addiction, tobacco dependence or addiction, reducing alcohol withdrawal symptoms or aiding in the cessation or lessening of alcohol use or substance abuse or behavioral dependencies.

2. The pharmaceutical composition according to claim 1, wherein said CB-1 receptor antagonist is selected from:
1-[9-(4-chloro-phenyl)-8-(2-chlorophenyl)-9H-purin-6-yl]-3-ethylamino-azetidine-3-carboxylic acid amide; 1-[9-(4-chloro-phenyl)-8-(2-chlorophenyl)-9H-purin-6-yl]-3-ethylamino-azetidine-3-carboxylic acid amide; 1-[9-(4-chlorophenyl)-8-(2-chlorophenyl)-9H-purin-6-yl]-3-isopropylaminoazetidine-3-carboxylic acid amide; 1-{1-[9-(4-chlorophenyl)-8-(2-chlorophenyl)-9H-purin-6-yl]-4-phenylpiperidin-4-yl}-ethanone; {3-[9-(4-chlorophenyl)-8-(2,4-dichlorophenyl)-9H-purin-6-yl]-3-(1α,5α,6α)-azabicyclo[3.1.0]hex-6-yl}-dimethylamine; 6-(1-benzylpyrrolidin-3-yloxy)-9-(4-chlorophenyl)-8-(2,4-dichlorophenyl)-9H-purine; 9-(4-chlorophenyl)-6-(1-cyclohexylazetidin-3-yloxy)-8-(2,4-dichlorophenyl)-9H-purine; 6-tert-butoxy-9-(4-chlorophenyl)-8-(2,4-dichlorophenyl)-9H-purine; 9-(4-chlorophenyl)-8-(2,4-dichlorophenyl)-6-isopropoxy-9H-purine; 1-[9-(4-chlorophenyl)-8-(2,4-dichlorophenyl)-9H-purin-6-yl]-4-propylaminopiperidine-4-carboxylic acid amide; 1-[9-(4-chlorophenyl)-8-(2-fluorophenyl)-9H-purin-6-yl]-4-propylaminopiperidine-4-carboxylic acid amide; 1-[9-(4-chlorophenyl)-8-(2-chlorophenyl)-9H-purin-6-yl]-4-propylaminopiperidine-4-carboxylic acid amide; 1-[9-(4-chlorophenyl)-8-(2-fluorophenyl)-2-methyl-9H-purin-6-yl]-4-isopropylaminopiperidine-4-carboxylic acid amide; 1-[9-(4-chlorophenyl)-8-(2-chlorophenyl)-9H-purin-6-yl]4-pyrrolidin-1-yl-piperidine-4-carboxylic acid amide; 1-[9-(4-chlorophenyl)-8-(2-chlorophenyl)-9H-purin-6-yl]-4-ethylamino-piperidine-4-carboxylic acid amide; 1-[9-(4-chlorophenyl)-8-(2-chlorophenyl)-9H-purin-6-yl]-4-isopropylaminopiperidine-4-carboxylic acid amide; 8-[9-(4-chlorophenyl)-8-(2-chlorophenyl)-9H-purin-6-yl]-1-isopropyl-1,3,8-triazaspiro[4.5]decan-4-one; 9-[9-(4-chlorophenyl)-8-(2-chlorophenyl)-9H-purin-6-yl]-1-methyl-4-oxa-1,9-diazaspiro[5.5]undecan-2-one; 8-[9-(4-chlorophenyl)-8-(2,4-dichlorophenyl)-9H-purin-6-yl]-1-isopropyl-1,3,8-triazaspiro[4.5]decan-4-one; 1-[9-(4-chlorophenyl)-8-(2-chlorophenyl)-9H-purin-6-yl]-4-(4-fluorophenyl)-piperidin-4-ol; 1-[9-(4-chlorophenyl)-8-(2-chlorophenyl)-9H-purin-6-yl]-4-phenylpiperidin-4-ol; 4-benzyl-1-[9-(4-chlorophenyl)-8-(2-chlorophenyl)-9H-purin-6-yl]-piperidin-4-ol; 4-[9-(4-chlorophenyl)-8-(2-chlorophenyl)-9H-purin-6-yl]-piperazine-2-carboxylic acid methylamide; 9-(4-chlorophenyl)-8-(2,4-dichlorophenyl)-6-(4-pyridin-2-yl-piperazin-1-yl)-9H-purine; and 9-(4-chlorophenyl)-8-(2,4-dichlorophenyl)-6-(4-pyrimidin-2-yl-piperazin-1-yl)-9H-purine; 1-[9-(4-chlorophenyl)-8-(2-fluorophenyl)-9H-purin-6-yl]-4-isopropylamino-piperidine-4-carboxylic acid amide; 1-[9-(4-chlorophenyl)-8-(2-chlorophenyl)-9H-purin-6-yl]-4-isopropylamino-piperidine-4-carboxylic acid amide; 4-amino-1-[9-(4-chlorophenyl)-8-(2-chlorophenyl)-9H-purin-6-yl]-piperidine-4-carboxylic acid amide; 1-[9-(4-chlorophenyl)-8-(2-chlorophenyl)-9H-purin-6-yl]-4-ethylamino-piperidine-4-carboxylic acid amide; 8-[9-(4-chlorophenyl)-8-(2-chlorophenyl)-9H-purin-6-yl]-1-isopropyl-1,3,8-triazaspiro[4.5]decan-4-one; 4-amino-1-[9-(4-chloro-phenyl)-8-(2-chlorophenyl)-9H-purin-6-yl]-piperidine-4-carboxylic acid amide; and 1-[9-(4-chlorophenyl )-8-(2-chlorophenyl)-9H-purin-6-yl]-4-ethylaminopiperidine-4-carboxylic acid amide; and a pharmaceutically acceptable salt thereof or a solvate or hydrate of said compound or said salt.

3. The pharmaceutical composition according to claim 1, wherein the CB-1 receptor antagonist is selected from:
7-(2-chlorophenyl)-8-(4-chlorophenyl)-2-methyl-4-(4-methylpiperazin-1-yl)-pyrazolo[1,5-a][1,3,5]triazine;
7-(2-chlorophenyl)-8-(4-chlorophenyl)-2-methyl-4-(4-pyrimidin-2-ylpiperazin-1-yl)-pyrazolo[1,5-a][1,3,5]triazine;
7-(2-chlorophenyl)-8-(4-chlorophenyl)-4-[(1S,4S)-5-methanesulfonyl-2,5-diazabicyclo[2.2.1]hept-2-yl]-2-methylpyrazolo[1,5-a][1,3,5]triazine; and
7-(2-chlorophenyl)-8-(4-chlorophenyl)-2-methyl-4-[4-(propane-2-sulfonyl )-piperazin-1-yl]-pyrazolo[1,5-a][1,3,5]triazine;
1-[7-(2-chlorophenyl)-8-(4-chlorophenyl )-2-methylpyrazolo[1,5-a][1,3,5]triazin-4-yl]-4-methylaminopiperidine-4-carboxylic acid amide;
1-[7-(2-chlorophenyl)-8-(4-fluorophenyl)-2-methylpyrazolo[1,5-a][1,3,5]triazin-4-yl]-4-ethylaminopiperidine-4-carboxylic acid amide;
1-[7-(2-chlorophenyl)-8-(4-chlorophenyl )-2-methylpyrazolo[1,5-a][1,3,5]triazin-4-yl]-4-ethylaminopiperidine-4-carboxylic acid amide;
1-[7-(2-chlorophenyl)-8-(4-chlorophenyl)-2-methylpyrazolo[1,5-a][1,3,5]triazin-4-yl]-4-isopropylaminopiperidine-4-carboxylic acid amide;
1-[7-(2-chlorophenyl)-8-(4-chlorophenyl )-2-methylpyrazolo[1,5-a][1,3,5]triazin-4-yl]-3-ethylaminoazetidine-3-carboxylic acid amide;
1-[7-(2-chlorophenyl)-8-(4-chlorophenyl)-2-methylpyrazolo[1,5-a][1,3,5]triazin-4-yl]-3-isopropylaminoazetidine-3-carboxylic acid amide;
3-amino-1-[7-(2-chlorophenyl)-8-(4-chlorophenyl)-2-methylpyrazolo[1,5-a][1,3,5]triazin-4-yl]-azetidine-3-carboxylic acid amide;
1-[7-(2-chlorophenyl)-8-(4-chlorophenyl)-2-methylpyrazolo[1,5-a][1,3,5]triazin-4-yl]-3-methylaminoazetidine-3-carboxylic acid amide; and
1-[7-(2-chlorophenyl)-8-(4-chlorophenyl)-2-methylpyrazolo[1,5-a][1,3,5]triazin-4-yl]-3-dimethylaminoazetidine-3-carboxylic acid amide;
1-{1-[7-(2-chlorophenyl)-8-(4-chlorophenyl)-2-methylpyrazolo[1,5-a][1,3,5]triazin-4-yl]-4-phenylpiperidin-4-yl}-ethanone;
3-[7-(2-chlorophenyl)-8-(4-chlorophenyl)-2-methylpyrazolo[1,5-a][1,3,5]triazin-4-yl]-3-azabicyclo[3.1.0]hex-6-ylamine;
1-[7-(2-chlorophenyl )-8-(4-chlorophenyl)-2-methylpyrazolo[1,5-a][1,3,5]triazin-4-yl]-4-(4-fluorophenyl)-piperidin-4-ol;
4-benzyl-1-[7-(2-chlorophenyl)-8-(4-chlorophenyl)-2-methylpyrazolo[1,5-a][1,3,5]triazin-4-yl]-piperidin-4-ol;
2-[7-(2-chlorophenyl )-8-(4-chlorophenyl )-2-methylpyrazolo[1,5-a][1,3,5]triazin-4-yl]-5-methyl-2,5,7-triazaspiro[3.4]octan-8-one;
2-[7-(2-chlorophenyl)-8-(4-chlorophenyl)-2-methylpyrazolo[1,5-a][1,3,5]triazin-4-yl]-2,5,7-triazaspiro[3.4]octan-8-one;
8-[7-(2-chlorophenyl)-8-(4-chlorophenyl)-2-methylpyrazolo[1,5-a][1,3,5]triazin-4-yl]-1-isopropyl-1,3,8-triazaspiro[4.5]decan-4-one;
2-[7-(2-chlorophenyl)-8-(4-chlorophenyl)-2-methylpyrazolo[1,5-a][1,3,5]triazin-4-yl]-6,6-dimethyl-2,5,7-triazaspiro[3.4]octan-8-one;
4-(1-benzylpyrrolidin-3-yloxy)-7-(2-chlorophenyl)-8-(4-chlorophenyl)-2-methylpyrazolo[1,5-a][1,3,5]triazine;
7-(2-chlorophenyl)-8-(4-chlorophenyl)-4-(1-cyclohexylazetidin-3-yloxy)-2-methylpyrazolo[1,5-a][1,3,5]triazine;
7-(2-chlorophenyl)-8-(4-chlorophenyl)-4-isopropoxy-2-methylpyrazolo[1,5-a][1,3,5]triazine; and
4-tert-butoxy-7-(2-chlorophenyl)-8-(4-chlorophenyl)-2-methylpyrazolo[1,5-a][1,3,5]triazine;
butyl-[7-(2-chlorophenyl)-8-(4-chlorophenyl)-2-methylpyrazolo[1,5-a][1,3,5]triazin-4-yl]-amine;
7-(2-chlorophenyl)-8-(4-chlorophenyl)-2-methyl-4-piperidin-1-yl-pyrazolo[1,5-a][1,3,5]triazine;
[7-(2-chlorophenyl)-8-(4-chlorophenyl)-2-methylpyrazolo[1,5-a][1,3,5]triazin-4-yl]-[2-(4-fluorophenyl)-ethyl]-amine;
7-(2-chlorophenyl)-8-(4-chlorophenyl)-2-methyl-4-morpholin-4-yl-pyrazolo[1,5-a][1,3,5]triazine; and
[7-(2-chlorophenyl)-8-(4-chlorophenyl)-2-methylpyrazolo[1,5-a][1,3,5]triazin-4-yl]-(2-morpholin-4-yl-ethyl)-amine;
1-[7-(2-chlorophenyl )-8-(4-chlorophenyl)-2-methylpyrazolo[1,5-a][1,3,5]triazin-4-yl]-4-ethylaminopiperidine-4-carboxylic acid amide;
1-[7-(2-chlorophenyl)-8-(4-chlorophenyl)-2-methylpyrazolo[1,5-a][1,3,5]triazin-4-yl]-3-ethylaminoazetidine-3-carboxylic acid amide;
1-[7-(2-chlorophenyl)-8-(4-chlorophenyl)-2-methylpyrazolo[1,5-a][1,3,5]triazin-4-yl]-3-isopropylaminoazetidine-3-carboxylic acid amide;
3-amino-1 -[7-(2-chlorophenyl)-8-(4-chlorophenyl)-2-methylpyrazolo[1,5-a][1,3,5]triazin-4-yl]-azetidine-3-carboxylic acid amide; and
8-[7-(2-chlorophenyl)-8-(4-chlorophenyl)-2-methylpyrazolo[1,5-a][1,3,5]triazin-4-yl]-1-isopropyl-1,3,8-triazaspiro[4.5]decan-4-one; and
a pharmaceutically acceptable salt thereof or a solvate or hydrate of said compound or said salt.

4. The pharmaceutical composition according to claim 1, wherein said CB-1 receptor antagonist is selected from:
3-(4-chlorophenyl)-2-(2-chlorophenyl)-7-(4-methyl-piperazin-1-yl)-pyrazolo[1,5-a]pyrimidine;
3-(4-chlorophenyl)-2-(2-chlorophenyl)-7-(4-pyrimidin-2-yl-piperazin-1-yl)-pyrazolo[1,5-a]pyrimidine;
3-(4-chloro-phenyl)-2-(2-chlorophenyl)-7-[(1S,4S)-5-methanesulfonyl-2,5-diazabicyclo[2.2.1]hept-2-yl]-pyrazolo[1,5-a]pyrimidine; and
3-(4-chlorophenyl)-2-(2-chlorophenyl)-7-[4-(propane-2-sulfonyl)-piperazin-1-yl]-pyrazolo[1,5-a]pyrimidine;
1-[3-(4-chlorophenyl)-2-(2-chlorophenyl)-pyrazolo[1,5-a]pyrimidin-7-yl]-4-ethylaminopiperidine-4-carboxylic acid amide;
1-[3-(4-chlorophenyl)-2-(2-chlorophenyl)-pyrazolo[1,5-a]pyrimidin-7-yl]-4-isopropylaminopiperidine-4-carboxylic acid amide;
1-[3-(4-chlorophenyl)-2-(2-chlorophenyl)-pyrazolo[1,5-a]pyrimidin-7-yl]-3-ethylaminoazetidine-3-carboxylic acid amide;
3-amino-1-[3-(4-chlorophenyl)-2-(2-chlorophenyl)-pyrazolo[1,5-a]pyrimidin-7-yl]-azetidine-3-carboxylic acid amide;
1-[3-(4-chlorophenyl)-2-(2-chlorophenyl)-6-methylpyrazolo[1,5-a]pyrimidin-7-yl]-3-ethylaminoazetidine-3-carboxylic acid amide;
1-[3-(4-chlorophenyl)-2-(2-chlorophenyl)-pyrazolo[1,5-a]pyrimidin-7-yl]-3-isopropylaminoazetidine-3-carboxylic acid amide;
1-[3-(4-chlorophenyl)-2-(2-chlorophenyl )-5,6-dimethylpyrazolo[1,5-a]pyrimidin-7-yl]-3-ethylamino-azetidine-3-carboxylic acid amide;
1-[3-(4-chlorophenyl)-2-(2-chlorophenyl)-pyrazolo[1,5-a]pyrimidin-7-yl]-3-methylaminoazetidine-3-carboxylic acid amide;
1-[3-(4-chlorophenyl)-2-(2-chlorophenyl )-5-methylpyrazolo[1,5-a]pyrimidin-7-yl]-3-ethylaminoazetidine-3-carboxylic acid amide;
1-{1-[3-(4-chlorophenyl)-2-(2-chlorophenyl)-pyrazolo[1,5-a]pyrimidin-7-yl]-4-phenylpiperidin-4-yl}-ethanone;
3-[3-(4-chlorophenyl)-2-(2-chlorophenyl)-pyrazolo[1,5-a]pyrimidin-7-yl]-3-(1a,5a,6a)-azabicyclo[3.1.0]hex-6-ylamine;
1-[3-(4-chlorophenyl)-2-(2-chlorophenyl )-pyrazolo[1,5-a]pyrimidin-7-yl]-4-(4-fluorophenyl)-piperidin-4-ol;
4-benzyl-1-[3-(4-chlorophenyl)-2-(2-chlorophenyl)-pyrazolo[1,5-a]pyrimidin-7-yl]-piperidin-4-ol;
8-[3-(4-chlorophenyl)-2-(2-chlorophenyl)-pyrazolo[1,5-a]pyrimidin-7-yl]-1-isopropyl-1,3,8-triazaspiro[4.5]decan-4-one;
2-[3-(4-chlorophenyl)-2-(2-chlorophenyl)-pyrazolo[1,5-a]pyrimidin-7-yl]-2,5,7-triazaspiro[3.4]octan-8-one;
8-[3-(4-chlorophenyl)-2-(2-chlorophenyl)-6-methylpyrazolo[1,5-a]pyrimidin-7-yl]-1-isopropyl-1,3,8-triazaspiro[4.5]decan-4-one;
2-[3-(4-chlorophenyl)-2-(2-chlorophenyl)-pyrazolo[1,5-a]pyrimidin-7-yl]-5-methyl-2,5,7-triazaspiro[3.4]octan-8-one;
7-(1-benzypyrrolidin-3-yloxy)-3-(4-chlorophenyl)-2-(2-chlorophenyl)-pyrazolo[1,5-a]pyrimidine; and
3-(4-chlorophenyl)-2-(2-chlorophenyl)-7-(1-cyclohexylazetidin-3-yloxy)-pyrazolo[1,5-a]pyrimidine;
1-[3-(4-chlorophenyl)-2-(2-chlorophenyl)-pyrazolo[1,5-a]pyrimidin-7-yl]4-ethylaminopiperidine-4-carboxylic acid amide;
1-[3-(4-chlorophenyl)-2-(2-chlorophenyl)-pyrazolo[1,5-a]pyrimidin-7-yl]-4-isopropylaminopiperidine-4-carboxylic acid amide; and
1-[3-(4-chlorophenyl)-2-(2-chlorophenyl)-pyrazolo[1,5-a]pyrimidin-7-yl]-3-ethylaminoazetidine-3-carboxylic acid amide; and
a pharmaceutically acceptable salt thereof or a solvate or hydrate of said compound or said salt.

5. The pharmaceutical composition according to claim 1, wherein said CB-1 receptor antagonist is selected from:
5-(4-chloro-phenyl)-3-(5-cyclohexyl-1H-imidazol-2-yl)-1-(2,4-dichloro-phenyl)-4-methyl-1H-pyrazole;
5-(4-chloro-phenyl)-3-(2-cyclohexyl-3H-imidazol-4-yl)-1-(2,4-dichloro-phenyl)-4-methyl-1H-pyrazole;
5-(4-chloro-phenyl)-1-(2,4-dichloro-phenyl)-4-methyl-3-[1-(1-methyl-1-phenyl-ethyl)-1H-imidazol-4-yl]-1H-pyrazole;
5-(4-chloro-phenyl)-1-(2-chloro-phenyl)-4-methyl-3-[1-(1-phenyl-ethyl)-1H-imidazol-4-yl]-1H-pyrazole;
5-(4-chloro-phenyl)-1-(2-fluoro-phenyl)-4-methyl-3-[1-(1-methyl-1-phenyl-ethyl)-1H-imidazol-4-yl]-1H-pyrazole;
5-(4-chloro-phenyl)-1-(2-chloro-phenyl)-3-[1-(2,2-dimethyl-tetrahydro-pyran-4-yl)-1H-imidazol-4-yl]-4-methyl-1H-pyrazole:
5-{2-(2,4-dichloro-phenyl)4-methyl-5-[1-(1-methyl-1-phenyl-ethyl)-1H-imidazol-4-yl]-2H-pyrazol-3-yl}-2-methoxy-pyridine; and
1-(2-chloro-phenyl)-5-(4-chloro-phenyl)-4-methyl-3-[1-(1-methyl-1-phenyl-ethyl)-1H-imidazol-4-yl]-1H-pyrazole; and
a pharmaceutically acceptable salt thereof or a solvate or hydrate of the compound or the salt.

6. The pharmaceutical composition according to claim 1, wherein said CB-1 receptor antagonist is selected from:
1-[5-(4-chloro-phenyl)-1-(2-chloro-phenyl)-4-methyl-1H-pyrazol-3-yl]-2-piperidin-1-yl-ethanone;
1-[5-(4-chloro-phenyl)-1-(2-chloro-phenyl)-4-methyl-1H-pyrazol-3-yl]-2-morpholin-4-yl-ethanone;
1-[5-(4-chloro-phenyl)-1-(2-chloro-phenyl)-4-methyl-1H-pyrazol-3-yl]-2-[4-(1-methyl-1H-pyrrole-2-carbonyl)-piperazin-1-yl]-ethanone;
1-[5-(4-chloro-phenyl)-1-(2-chloro-phenyl)4-methyl-1H-pyrazol-3-yl]-2-[4-(1-methyl-cyclopropanecarbonyl)-piperazin-1-yl]-ethanone;
N-(1-{2-[5-(4-chloro-phenyl)-1-(2-chloro-phenyl)-4-methyl-1H-pyrazol-3-yl]-2-oxo-ethyl}-piperidin-4-yl)-2,2,2-trifluoro-acetamide;
1-[5-(4-chloro-phenyl)-1-(2-fluoro-phenyl)-4-methyl-1H-pyrazol-3-yl]-2-morpholin-4-yl-ethanone;
1-[5-(4-chloro-phenyl)-1-(2-fluoro-phenyl )-4-methyl-1H-pyrazol-3-yl]-2-piperidin-1-yl-ethanone;
1-[5-(4-chloro-phenyl)-1-(2-fluoro-phenyl )-4-methyl-1H-pyrazol-3-yl]-2-(4-trifluoroacetyl-piperazin-1-yl)-ethanone;
1-[1-(2-chloro-phenyl)-5-(4-chloro-phenyl)-4-methyl-1H-pyrazol-3-yl]-2-pyrrolidin-1-yl-ethanone;
1-[1-(2-chloro-phenyl)-5-(4-chloro-phenyl)-4-methyl-1H-pyrazol-3-yl]-2-[1,4]oxazepan-4-yl-ethanone; and
1-[5-(4-chloro-phenyl)-1-(2-chloro-phenyl)-4-methyl-1H-pyrazol-3-yl]-2-(1-oxa-8-aza-spiro[4.5]dec-8-yl)-ethanone; and
a pharmaceutically acceptable salt thereof, or a solvate or hydrate of the compound.

7. The pharmaceutical composition according claim 1, wherein said CB-1 receptor antagonist is selected from:
2-(benzyl-isopropyl-amino)-1-[1-(2-chloro-phenyl)-5-(4-chloro-phenyl)-4-methyl-1H-pyrazol-3-yl]-ethanol;
1-[5-(4-chloro-phenyl)-1-(2-chloro-phenyl)-4-methyl-1H-pyrazol-3-yl]-2-(3,5-dimethyl-piperidin-1-yl)-ethanol;
1-{2-[1-(2-chloro-phenyl)-5-(4-chloro-phenyl)-4-methyl-1H-pyrazol-3-yl]-2-hydroxy-ethyl}-4-isopropylamino-piperidine-4-carboxylic acid amide;
1-[5-(4-chloro-phenyl)-1-(2-chloro-phenyl)-4-methyl-1H-pyrazol-3-yl]-2-(3,3-dimethyl-piperidin-1-yl)-ethanol;
1-[5-(4-chloro-phenyl)-1-(2-chloro-phenyl)-4-methyl-1H-pyrazol-3-yl]-2-piperidin-1-yl-ethanol; and
1-[5-(4-chloro-phenyl)-1-(2-chloro-phenyl)4-methyl-1H-pyrazol-3-yl]-2-morpholin-4-yl-ethanol; and
a pharmaceutically acceptable salt thereof, or a solvate or hydrate of the compound.

8. The pharmaceutical composition according to claim 1 wherein said CB-1 receptor antagonist is selected from:
2-[5-(4-chloro-phenyl)-1-(2-chloro-phenyl)-4-methyl-1H-pyrazol-3-yl]-4-cyclohexyl-morpholine;
2-[5-(4-chloro-phenyl)-1-(2-chloro-phenyl)-4-methyl-1H-pyrazol-3-yl]-4-(propane-2-sulfonyl)-morpholine;
2-[5-(4-chloro-phenyl)-1-(2-chloro-phenyl)-4-methyl-1H-pyrazol-3-yl]-4-(toluene-4-sulfonyl)-morpholine;
1-{2-[1-(2-chloro-phenyl)-5-(4-chloro-phenyl)-4-methyl-1H-pyrazol-3-yl]-morpholin-4-yl}-2-methyl-propan-1-one; and
2-[1-(2-chloro-phenyl)-5-(4-chloro-phenyl)-4-methyl-1H-pyrazol-3-yl]-4-(4-trifluoromethyl-benzyl)-morpholine; and
a pharmaceutically acceptable salt thereof or a solvate or hydrate of the compound.

9. The pharmaceutical composition according claim 1, wherein said CB-1 receptor antagonist is selected from:
1-[1-(4-chloro-phenyl)-2-(2,4-dichloro-phenyl)-5-methyl-1H-imidazol-4-yl]-2-piperidin-1-yl-ethanone and
1-[1-(4-chloro-phenyl)-2-(2,4-dichloro-phenyl)-5-methyl-1H-imidazol-4-yl]-2-morpholin-4-yl-ethanone; and
a pharmaceutically acceptable salt thereof, or a solvate or hydrate of the compound.

10. The pharmaceutically composition according to claim 1, wherein said opioid receptor antagonist is selected from:
2-methoxy-ethanesulfonic acid {3-[6-ethyl-3-(2-hydroxy-1,2,3,4-tetrahydro-naphthalen-2-ylmethyl)-3-aza-bicyclo[3.1.0]hex-6-yl]-phenyl}-amide;
N-[3-(6-ethyl-3-indan-2-ylmethyl-3-aza-bicyclo[3.1.0]hex-6-yl)-phenyl]-methanesulfonamide;
2-methoxy-ethanesulfonic acid [3-(6-ethyl-3-indan-2-ylmethyl-3-aza-bicyclo[3.1.0]hex-6-yl)-phenyl]-amide;
N-{3-[6-ethyl-3-(2-hydroxy-indan-2-ylmethyl)-3-aza-bicyclo[3.1.0]hex-6-yl]-phenyl}-methanesulfonamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
N-(3-{6-ethyl-3-[3-(1-hydroxy-cyclohexyl )-propyl]-3-aza-bicyclo[3.1.0] hex-6-yl}-phenyl)-methanesulfonamide;
3-{3-[3-(1-hydroxy-cyclohexyl)-propyl]-8-methoxy-3-aza-bicyclo[3.2.1]oct-8-yl}-benzamide;
2-methoxy-ethanesulfonic acid {3-[6-ethyl-3-(2-hydroxy-indan-2-ylmethyl)-3-aza-bicyclo[3.1.0]hex-6-yl]-phenyl}-amide;
3-[6-ethyl-3-(2-hydroxy-indan-2-ylmethyl)-3-aza-bicyclo[3.1.0]hex-6-yl]-benzamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
3-[6-ethyl-3-(2-hydroxy-1,2,3,4-tetrahydro-naphthalen-2-ylmethyl)-3-aza-bicyclo[3.1.0]hex-6-yl]-benzamide;
N-{3-[2-(2-hydroxy-indan-2-ylmethyl)-2-aza-bicyclo[3.3.1]non-5-yl]-phenyl}-methanesulfonamide;
2-methoxy-ethanesulfonic acid {3-[3-(2-hydroxy-indan-2-ylmethyl)-8-methoxy-3-aza-bicyclo[3.2.1]oct-8-yl]-phenyl}-amide;
3-{6-ethyl-3-[3-(1-hydroxy-cyclohexyl)-propyl]-3-aza-bicyclo[3.1.0]hex-6-yl}-benzamide;
3-[6-ethyl-3-(1-hydroxy-3-phenyl-cyclobutylmethyl)-3-aza-bicyclo[3.1.0]hex-6-yl]-benzamide;
2-methoxy-ethanesulfonic acid (3-{6-ethyl-3-[3-(1-hydroxy-cyclohexyl)-propyl]-3-aza-bicyclo[3.1.0]hex-6-yl}-phenyl)-amide;
3-{1-[3-(1-hydroxy-cyclohexyl)-propyl]-3,4-dimethyl-piperidin-4-yl}-benzamide;
3-(1-indan-2-ylmethyl-3,4-dimethyl-piperidin-4-yl)-benzamide;
N-{3-[3-(2-hydroxy-indan-2-ylmethyl)-8-methoxy-3-aza-bicyclo[3.2.1]oct-8-yl]-phenyl}-methanesulfonamide;
3-[1-(1-hydroxy-3-phenyl-cyclobutylmethyl)-3,4-dimethyl-piperidin-4-yl]-benzamide;
3-(6-hthyl-3-indan-2-ylmethyl-3-aza-bicyclo[3.1.0]hex-6-yl)-benzamide;
N-(3-{3-[3-(1-hydroxy-cyclohexyl)-propyl]-8-methoxy-3-aza-bicyclo[3.2.1]oct-8-yl}-phenyl)-methanesulfonamide;
3-[1 -(2-hydroxy-indan-2-ylmethyl)-3,4-dimethyl-piperidin-4-yl]-benzamide;
N-(3-{2-[3-(1-hydroxy-cyclohexyl)-propyl]-2-aza-bicyclo[3.3.1]non-5-yl}-phenyl)-methanesulfonamide;
3-[3-(1-hydroxy-3-phenyl-cyclobutylmethyl)-8-methoxy-3-aza-bicyclo[3.2.1]oct-8-yl]-benzamide;
3-[3-(2-hydroxy-indan-2-ylmethyl)-8-methoxy-3-aza-bicyclo[3.2.1]oct-8-yl]-benzamide;
3-[2-(2-hydroxy-indan-2-ylmethyl)-2-aza-bicyclo[3.3.1]non-5-yl]-benzamide;
3-{2-[3-(1-hydroxy-cyclohexyl)-propyl]-2-aza-bicyclo[3.3.1]non-5-yl}-benzamide;
3-[2-(1-hydroxy-3-phenyl-cyclobutylmethyl)-2-aza-bicyclo[3.3.1]non-5-yl]-benzamide;
3-[2-(2-hydroxy-1,2,3,4-tetrahydro-naphthalen-2-ylmethyl)-2-aza-bicyclo[3.3.1]non-5-yl]-benzamide;
2-methoxy-ethanesulfonic acid {3-[2-(2-hydroxy-indan-2-ylmethyl )-2-aza-bicyclo[3.3.1]non-5-yl]-phenyl}-amide;
2-methoxy-ethanesulfonic acid (3-{2-[3-(1-hydroxy-cyclohexyl)-propyl]-2-aza-bicyclo[3.3.1]non-5-yl}-phenyl)-amide;
2-methoxy-ethanesulfonic acid {3-[2-(1-hydroxy-3-phenyl-cyclobutylmethyl)-2-aza-bicyclo[3.3.1]non-5-yl]-phenyl}-amide;
2-methoxy-ethanesulfonic acid {3-[2-(2-hydroxy-1,2,3,4-tetrahydro-naphthalen-2-ylmethyl)-2-aza-bicyclo[3.3.1]non-5-yl]-phenyl}-amide;
N-{3-[2-(1-hydroxy-3-phenyl-cyclobutylmethyl)-2-aza-bicyclo[3.3.1]non-5-yl]-phenyl}-methanesulfonamide; and
N-{3-[2-(2-hydroxy-1,2,3,4-tetrahydro-naphthalen-2-ylmethyl)-2-aza-bicyclo[3.3.1]non-5-yl]-phenyl}-methanesulfonamide.
Preferably, the opioid receptor antagonist is selected from:
2-methoxy-ethanesulfonic acid {3-[6-ethyl-3-(2-hydroxy-1,2,3,4-tetrahydro-naphthalen-2-ylmethyl)-3-aza-bicyclo[3.1.0]hex-6-yl]-phenyl}-amide;
N-[3-(6-ethyl-3-indan-2-ylmethyl-3-aza-bicyclo[3.1.0]hex-6-yl)-phenyl]-methanesulfonamide;
2-methoxy-ethanesulfonic acid [3-(6-ethyl-3-indan-2-ylmethyl-3-aza-bicyclo[3.1.0]hex-6-yl)-phenyl]-amide;
N-{3-[6-ethyl-3-(2-hydroxy-indan-2-ylmethyl)-3-aza-bicyclo[3.1.0]hex-6-yl]-phenyl}-methanesulfonamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
N-(3-{6-ethyl-3-[3-(1-hydroxy-cyclohexyl)-propyl]-3-aza-bicyclo[3.1.0]hex-6-yl}-phenyl)-methanesulfonamide;
3-{3-[3-(1-hydroxy-cyclohexyl)-propyl]-8-methoxy-3-aza-bicyclo[3.2.1]oct-8-yl}-benzamide;
2-methoxy-ethanesulfonic acid {3-[6-ethyl-3-(2-hydroxy-indan-2-ylmethyl)-3-aza-bicyclo[3.1.0]hex-6-yl]-phenyl}-amide;
3-[6-ethyl-3-(2-hydroxy-indan-2-ylmethyl)-3-aza-bicyclo[3.1.0]hex-6-yl]-benzamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
3-[6-ethyl-3-(2-hydroxy-1,2,3,4-tetrahydro-naphthalen-2-ylmethyl)-3-aza-bicyclo[3.1.0]hex-6-yl]-benzamide;
N-{3-[2-(2-hydroxy-indan-2-ylmethyl)-2-aza-bicyclo[3.3.1]non-5-yl]-phenyl}-methanesulfonamide;
2-methoxy-ethanesulfonic acid {3-[3-(2-hydroxy-indan-2-ylmethyl)-8-methoxy-3-aza-bicyclo[3.2.1]oct-8-yl]-phenyl}-amide;
3-{6-ethyl-3-[3-(1-hydroxy-cyclohexyl )-propyl]-3-aza-bicyclo[3.1.0]hex-6-yl}-benzamide;
3-[6-ethyl-3-(1-hydroxy-3-phenyl-cyclobutylmethyl)-3-aza-bicyclo[3.1.0]hex-6-yl]-benzamide;
2-methoxy-ethanesulfonic acid (3-{6-ethyl-3-[3-(1-hydroxy-cyclohexyl)-propyl]-3-aza-bicyclo[3.1.0]hex-6-yl}-phenyl)-amide;
3-{1-[3-(1-hydroxy-cyclohexyl)-propyl]-3,4-dimethyl-piperidin-4-yl}-benzamide;
3-(1-indan-2-ylmethyl-3,4-dimethyl-piperidin-4-yl)-benzamide;
N-{3-[3-(2-hydroxy-indan-2-ylmethyl)-8-methoxy-3-aza-bicyclo[3.2.1]oct-8-yl]-phenyl}-methanesulfonamide;
3-[1 -(1-hydroxy-3-phenyl-cyclobutylmethyl)-3,4-dimethyl-piperidin-4-yl]-benzamide;
3-(6-hthyl-3-indan-2-ylmethyl-3-aza-bicyclo[3.1.0]hex-6-yl)-benzamide;
N-(3-{3-[3-(1-hydroxy-cyclohexyl)-propyl]-8-methoxy-3-aza-bicyclo[3.2.1]oct-8-yl}-phenyl)-methanesulfonamide;
3-[1 -(2-hydroxy-indan-2-ylmethyl)-3,4-dimethyl-piperidin-4-yl]-benzamide;
N-(3-{2-[3-(1-hydroxy-cyclohexyl)-propyl]-2-aza-bicyclo[3.3.1]non-5-yl}-phenyl)-methanesulfonamide;
3-[3-(1-hydroxy-3-phenyl-cyclobutylmethyl)-8-methoxy-3-aza-bicyclo[3.2.1]oct-8-yl]-benzamide;
3-[3-(2-hydroxy-indan-2-yl methyl)-8-methoxy-3-aza-bicyclo[3.2.1]oct-8-yl]-benzamide;
3-[2-(2-hydroxy-indan-2-ylmethyl)-2-aza-bicyclo[3.3.1]non-5-yl]-benzamide;
3-{2-[3-(1-hydroxy-cyclohexyl)-propyl]-2-aza-bicyclo[3.3.1]non-5-yl}-benzamide;
3-[2-(1-hydroxy-3-phenyl-cyclobutylmethyl )-2-aza-bicyclo[3.3.1]non-5-yl]-benzamide;
3-[2-(2-hydroxy-1,2,3,4-tetrahydro-naphthalen-2-ylmethyl)-2-aza-bicyclo[3.3.1]non-5-yl]-benzamide;
2-methoxy-ethanesulfonic acid {3-[2-(2-hydroxy-indan-2-ylmethyl)-2-aza-bicyclo[3.3.1]non-5-yl]-phenyl}-amide;
2-methoxy-ethanesulfonic acid (3-{2-[3-(1-hydroxy-cyclohexyl)-propyl]-2-aza-bicyclo[3.3.1]non-5-yl}-phenyl)-amide;
2-methoxy-ethanesulfonic acid {3-[2-(1-hydroxy-3-phenyl-cyclobutylmethyl)-2-aza-bicyclo[3.3.1]non-5-yl]-phenyl}-amide;
2-methoxy-ethanesulfonic acid {3-[2-(2-hydroxy-1,2,3,4-tetrahydro-naphthalen-2-ylmethyl)-2-aza-bicyclo[3.3.1]non-5-yl]-phenyl}-amide;
N-{3-[2-(1-hydroxy-3-phenyl-cyclobutylmethyl)-2-aza-bicyclo[3.3.1]non-5-yl]-phenyl}-methanesulfonamide; and
N-{3-[2-(2-hydroxy-1,2,3,4-tetrahydro-naphthalen-2-ylmethyl)-2-aza-bicyclo[3.3.1]non-5-yl]-phenyl}-methanesulfonamide.

11. The pharmaceutical composition according to claim 1 wherein said opioid receptor antagonist is selected from:
2-methoxy-ethanesulfonic acid {3-[6-ethyl-3-(2-hydroxy-1,2,3,4-tetrahydro-naphthalen-2-ylmethyl)-3-aza-bicyclo[3.1.0]hex-6-yl]-phenyl}-amide;
N-[3-(6-ethyl-3-indan-2-ylmethyl-3-aza-bicyclo[3.1.0]hex-6-yl)-phenyl]-methanesulfonamide;
2-methoxy-ethanesulfonic acid [3-(6-ethyl-3-indan-2-yl methyl-3-aza-bicyclo[3.1.0]hex-6-yl)-phenyl]-amide;
N-{3-[6-ethyl-3-(2-hydroxy-indan-2-ylmethyl)-3-aza-bicyclo[3.1.0]hex-6-yl]-phenyl}-methanesulfonamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
N-(3-{6-ethyl-3-[3-(1-hydroxy-cyclohexyl)-propyl]-3-aza-bicyclo[3.1.0]hex-6-yl}-phenyl)-methanesulfonamide;
3-{3-[3-(1-hydroxy-cyclohexyl)-propyl]-8-methoxy-3-aza-bicyclo[3.2.1]oct-8-yl}-benzamide;
2-methoxy-ethanesulfonic acid {3-[6-ethyl-3-(2-hydroxy-indan-2-ylmethyl)-3-aza-bicyclo[3.1.0]hex-6-yl]-phenyl}-amide;
3-[6-ethyl-3-(2-hydroxy-indan-2-ylmethyl)-3-aza-bicyclo[3.1.0]hex-6-yl]-benzamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
3-[6-ethyl-3-(2-hydroxy-1,2,3,4-tetrahydro-naphthalen-2-ylmethyl )-3-aza-bicyclo[3.1.0]hex-6-yl]-benzamide;
N-{3-[2-(2-hydroxy-indan-2-ylmethyl)-2-aza-bicyclo[3.3.1]non-5-yl]-phenyl}-methanesulfonamide;
2-methoxy-ethanesulfonic acid {3-[3-(2-hydroxy-indan-2-ylmethyl)-8-methoxy-3-aza-bicyclo[3.2.1]oct-8-yl]-phenyl}-amide;
3-{6-ethyl-3-[3-(1-hydroxy-cyclohexyl)-propyl]-3-aza-bicyclo[3.1.0]hex-6-yl}-benzamide;
3-[6-ethyl-3-(1-hydroxy-3-phenyl-cyclobutylmethyl )-3-aza-bicyclo[3.1.0]hex-6-yl]-benzamide;
2-methoxy-ethanesulfonic acid (3-{6-ethyl-3-[3-(1-hydroxy-cyclohexyl)-propyl]-3-aza-bicyclo[3.1.0]hex-6-yl}-phenyl)-amide;
3-{1 -[3-(1-hydroxy-cyclohexyl)-propyl]-3,4-dimethyl-piperidin-4-yl}-benzamide;
3-(1-indan-2-ylmethyl-3,4-dimethyl-piperidin-4-yl)-benzamide;
N-{3-[3-(2-hydroxy-indan-2-ylmethyl)-8-methoxy-3-aza-bicyclo[3.2.1]oct-8-yl]-phenyl}-methanesulfonamide;
3-[1 -(1-hydroxy-3-phenyl-cyclobutylmethyl)-3,4-dimethyl-piperidin-4-yl]-benzamide;
3-(6-hthyl-3-indan-2-ylmethyl-3-aza-bicyclo[3.1.0]hex-6-yl)-benzamide;
N-(3-{3-[3-(1-hydroxy-cyclohexyl)-propyl]-8-methoxy-3-aza-bicyclo[3.2.1]oct-8-yl}-phenyl)-methanesulfonamide;
3-[1 -(2-hydroxy-indan-2-yl methyl)-3,4-dimethyl-piperidin-4-yl]-benzamide;
N-(3-{2-[3-(1-hydroxy-cyclohexyl)-propyl]-2-aza-bicyclo[3.3.1]non-5-yl}-phenyl)-methanesulfonamide;
3-[3-(1-hydroxy-3-phenyl-cyclobutylmethyl)-8-methoxy-3-aza-bicyclo[3.2.1]oct-8-yl]-benzamide;
3-[3-(2-hydroxy-indan-2-yl methyl)-8-methoxy-3-aza-bicyclo[3.2.1]oct-8-yl]-benzamide;
3-[2-(2-hydroxy-indan-2-ylmethyl)-2-aza-bicyclo[3.3.1]non-5-yl]-benzamide;
3-{2-[3-(1-hydroxy-cyclohexyl)-propyl]-2-aza-bicyclo[3.3.1]non-5-yl}-benzamide;
3-[2-(1-hydroxy-3-phenyl-cyclobutylmethyl)-2-aza-bicyclo[3.3.1]non-5-yl]-benzamide;
3-[2-(2-hydroxy-1,2,3,4-tetrahydro-naphthalen-2-ylmethyl)-2-aza-bicyclo[3.3.1]non-5-yl]-benzamide;
2-methoxy-ethanesulfonic acid {3-[2-(2-hydroxy-indan-2-ylmethyl)-2-aza-bicyclo[3.3.1]non-5-yl]-phenyl}-amide;
2-methoxy-ethanesulfonic acid (3-{2-[3-(1-hydroxy-cyclohexyl)-propyl]-2-aza-bicyclo[3.3.1]non-5-yl}-phenyl)-amide;
2-methoxy-ethanesulfonic acid {3-[2-(1-hydroxy-3-phenyl-cyclobutylmethyl)-2-aza-bicyclo[3.3.1]non-5-yl]-phenyl}-amide;
2-methoxy-ethanesulfonic acid {3-[2-(2-hydroxy-1,2,3,4-tetrahydro-naphthalen-2-ylmethyl)-2-aza-bicyclo[3.3.1]non-5-yl]-phenyl}-amide;
N-{3-[2-(1-hydroxy-3-phenyl-cyclobutylmethyl)-2-aza-bicyclo[3.3.1]non-5-yl]-phenyl}-methanesulfonamide; and
N-{3-[2-(2-hydroxy-1,2,3,4-tetrahydro-naphthalen-2-ylmethyl)-2-aza-bicyclo[3.3.1]non-5-yl]-phenyl}-methanesulfonamide.

12. A method of treating a mammal which presents with alcohol, cocaine or nicotine addiction, alcohol withdrawal symptoms, substance abuse or behavioral dependencies including gambling, comprising administering to said mammal:
a. opioid receptor antagonist or a pharmaceutically acceptable salt thereof;
b. a CB-1 receptor antagonist or a pharmaceutically acceptable salt thereof, and
c. a pharmaceutically acceptable salt thereof;
wherein the opioid receptor antagonist and the CB-1 receptor antagonist are present in amounts that render the composition effective in the treatment of alcohol, cocaine or nicotine addiction, alcohol withdrawals symptoms, substance abuse or behavior dependencies.

13. The method according to claim 12, wherein said CB-1 receptor antagonist is selected from:
1-[9-(4-chloro-phenyl)-8-(2-chlorophenyl)-9H-purin-6-yl]-3-ethylamino-azetidine-3-carboxylic acid amide;
1-[9-(4-chloro-phenyl)-8-(2-chlorophenyl)-9H-purin-6-yl]-3-ethylamino-azetidine-3-carboxylic acid amide;
1-[9-(4-chlorophenyl)-8-(2-chlorophenyl)-9H-purin-6-yl]-3-isopropylaminoazetidine-3-carboxylic acid amide;
1-{1-[9-(4-chlorophenyl)-8-(2-chlorophenyl)-9H-purin-6-yl]-4-phenylpiperidin-4-yl}-ethanone;
{3-[9-(4-chlorophenyl)-8-(2,4-dichlorophenyl)-9H-purin-6-yl]-3-(1α,5α,6α)-azabicyclo[3.1.0]hex-6-yl}-dimethylamine;
6-(1-benzylpyrrolidin-3-yloxy)-9-(4-chlorophenyl)-8-(2,4-dichlorophenyl)-9H-purine; 9-(4-chlorophenyl)-6-(1-cyclohexylazetidin-3-yloxy)-8-(2,4-dichlorophenyl)-9H-purine;
6-tert-butoxy-9-(4-chlorophenyl)-8-(2,4-dichlorophenyl)-9H-purine;
9-(4-chlorophenyl)-8-(2,4-dichlorophenyl)-6-isopropoxy-9H-purine;
1-[9-(4-chlorophenyl )-8-(2,4-dichlorophenyl)-9H-purin-6-yl]-4-propylaminopiperidine-4-carboxylic acid amide;
1-[9-(4-chlorophenyl)-8-(2-fluorophenyl)-9H-purin-6-yl]-4-propylaminopiperidine-4-carboxylic acid amide;
1-[9-(4-chlorophenyl)-8-(2-chlorophenyl)-9H-purin-6-y]-4-propylaminopiperidine-4-carboxylic acid amide;
1-[9-(4-chlorophenyl)-8-(2-fluorophenyl)-2-methyl-9H-purin-6-yl]-4-isopropylaminopiperidine-4-carboxylic acid amide;
1-[9-(4-chlorophenyl)-8-(2-chlorophenyl)-9H-purin-6-yl]-4-pyrrolidin-1-yl-piperidine-4-carboxylic acid amide;
1-[9-(4-chlorophenyl)-8-(2-chlorophenyl)-9H-purin-6-yl]-4-ethylamino-piperidine-4-carboxylic acid amide;
1-[9-(4-chlorophenyl)-8-(2-chlorophenyl)-9H-purin-6-yl]-4-isopropylaminopiperidine-4-carboxylic acid amide;
8-[9-(4-chlorophenyl)-8-(2-chlorophenyl)-9H-purin-6-yl]-1-isopropyl-1,3,8-triazaspiro[4.5]decan-4-one;
9-[9-(4-chlorophenyl)-8-(2-chlorophenyl)-9H-purin-6-yl]-1-methyl-4-oxa-1,9-diazaspiro[5.5]undecan-2-one;
8-[9-(4-chlorophenyl)-8-(2,4-dichlorophenyl)-9H-purin-6-yl]-1-isopropyl-1,3,8-triazaspiro[4.5]decan-4-one;
1-[9-(4-chlorophenyl)-8-(2-chlorophenyl)-9H-purin-6-yl]-4-(4-fluorophenyl)-piperidin-4-ol;
1-[9-(4-chlorophenyl)-8-(2-chlorophenyl)-9H-purin-6-yl]-4-phenypiperidin-4-ol;
4-benzyl-1-[9-(4-chlorophenyl)-8-(2-chlorophenyl)-9H-purin-6-yl]-piperidin-4-ol;
4-[9-(4-chlorophenyl)-8-(2-chlorophenyl )-9H-purin-6-yl]-piperazine-2-carboxylic acid methylamide;
9-(4-chlorophenyl)-8-(2,4-dichlorophenyl)-6-(4-pyridin-2-yl-piperazin-1-yl)-9H-purine; and
9-(4-chlorophenyl)-8-(2,4-dichlorophenyl)-6-(4-pyrimidin-2-yl-piperazin-1-yl)-9H-purine;
1-[9-(4-chlorophenyl)-8-(2-fluorophenyl)-9H-purin-6-yl]-4-isopropylamino-piperidine-4-carboxylic acid amide;
1-[9-(4-chlorophenyl)-8-(2-chlorophenyl)-9H-purin-6-yl]-4-isopropylamino-piperidine-4-carboxylic acid amide;
4-amino-1-[9-(4-chlorophenyl)-8-(2-chlorophenyl)-9H-purin-6-yl]-piperidine-4-carboxylic acid amide;
1-[9-(4-chlorophenyl)-8-(2-chlorophenyl)-9H-purin-6-yl]-4-ethylamino-piperidine-4-carboxylic acid amide;
8-[9-(4-chlorophenyl)-8-(2-chlorophenyl)-9H-purin-6-yl]-1-isopropyl-1,3,8-triazaspiro[4.5]decan-4-one;
4-amino-1-[9-(4-chloro-phenyl)-8-(2-chlorophenyl)-9H-purin-6-yl]-piperidine-4-carboxylic acid amide; and
1-[9-(4-chlorophenyl)-8-(2-chlorophenyl)-9H-purin-6-yl]-4-ethylaminopiperidine-4-carboxylic acid amide; and
a pharmaceutically acceptable salt thereof of a solvate or hydrate of said compound or said salt.

14. The method according to claim 12, wherein the CB-1 receptor antagonist is selected from:
7-(2-chlorophenyl)-8-(4-chlorophenyl)-2-methyl-4-(4-methylpiperazin-1-yl)-pyrazolo[1,5-a][1,3,5]triazine;
7-(2-chlorophenyl )-8-(4-chlorophenyl)-2-methyl-4-(4-pyrimidin-2-ylpiperazin-1-yl)-pyrazolo[1,5-a][1,3,5]triazine;
7-(2-chlorophenyl)-8-(4-chlorophenyl)-4-[(1S,4S)-5-methanesulfonyl-2,5-diazabicyclo[2.2.1]hept-2-yl]-2-methylpyrazolo[1,5-a][1,3,5]triazine; and
7-(2-chlorophenyl)-8-(4-chlorophenyl)-2-methyl-4-[4-(propane-2-sulfonyl)-piperazin-1-yl]-pyrazolo[1,5-a][1,3,5]triazine;
1-[7-(2-chlorophenyl )-8-(4-chlorophenyl)-2-methylpyrazolo[1,5-a][1,3,5]triazin-4-yl]-4-methylaminopiperidine-4-carboxylic acid amide;
1-[7-(2-chlorophenyl)-8-(4-fluorophenyl )-2-methylpyrazolo[1,5-a][1,3,5]triazin-4-yl]-4-ethylaminopiperidine-4-carboxylic acid amide;
1-[7-(2-chlorophenyl)-8-(4-chlorophenyl )-2-methylpyrazolo[1,5-a][1,3,5]triazin-4-yl]-4-ethylaminopiperidine-4-carboxylic acid amide;
1-[7-(2-chlorophenyl)-8-(4-chlorophenyl)-2-methylpyrazolo[1,5-a][1,3,5]triazin-4-yl]-4-isopropylaminopiperidine-4-carboxylic acid amide;
1-[7-(2-chlorophenyl)-8-(4-chlorophenyl )-2-methylpyrazolo[1,5-a][1,3,5]triazin-4-yl]-3-ethylaminoazetidine-3-carboxylic acid amide;
1-[7-(2-chlorophenyl)-8-(4-chlorophenyl)-2-methylpyrazolo[1,5-a][1,3,5]triazin-4-yl]-3-isopropylaminoazetidine-3-carboxylic acid amide;
3-amino-1-[7-(2-chlorophenyl)-8-(4-chlorophenyl)-2-methylpyrazolo[1,5-a][1,3,5]triazin-4-yl]-azetidine-3-carboxylic acid amide;
1-[7-(2-chlorophenyl)-8-(4-chlorophenyl)-2-methylpyrazolo[1,5-a][1,3,5]triazin-4-yl]-3-methylaminoazetidine-3-carboxylic acid amide; and
1-[7-(2-chlorophenyl)-8-(4-chlorophenyl )-2-methylpyrazolo[1,5-a][1,3,5]triazin-4-yl]-3-dimethylaminoazetidine-3-carboxylic acid amide;
1-{1-[7-(2-chlorophenyl)-8-(4-chlorophenyl)-2-methylpyrazolo[1,5-a][1,3,5]triazin-4-yl]-4-phenylpiperidin-4-yl}-ethanone;
3-[7-(2-chlorophenyl)-8-(4-chlorophenyl)-2-methylpyrazolo[1,5-a][1,3,5]triazin-4-yl]-3-azabicyclo[3.1.0]hex-6-ylamine;
1-[7-(2-chlorophenyl)-8-(4-chlorophenyl )-2-methylpyrazolo[1,5-a][1,3,5]triazin-4-yl]-4-(4-fluorophenyl)-piperidin-4-ol;
4-benzyl-1 -[7-(2-chlorophenyl)-8-(4-chlorophenyl)-2-methylpyrazolo[1,5-a][1,3,5]triazin-4-yl]-piperidin-4-ol;
2-[7-(2-chlorophenyl)-8-(4-chlorophenyl )-2-methylpyrazolo[1,5-a][1,3,5]triazin-4-yl]-5-methyl-2,5,7-triazaspiro[3.4]octan-8-one;
2-[7-(2-chlorophenyl)-8-(4-chlorophenyl)-2-methylpyrazolo[1,5-a][1,3,5]triazin-4-yl]-2,5,7-triazaspiro[3.4]octan-8-one;
8-[7-(2-chlorophenyl)-8-(4-chlorophenyl)-2-methylpyrazolo[1,5-a][1,3,5]triazin-4-yl]-1-isopropyl-1,3,8-triazaspiro[4.5]decan-4-one;
2-[7-(2-chlorophenyl)-8-(4-chlorophenyl)-2-methylpyrazolo[1,5-a][1,3,5]triazin-4-yl]-6,6-dimethyl-2,5,7-triazaspiro[3.4]octan-8-one;
4-(1-benzylpyrrolidin-3-yloxy)-7-(2-chlorophenyl)-8-(4-chlorophenyl)-2-methylpyrazolo[1,5-a][1,3,5]triazine;
7-(2-chlorophenyl)-8-(4-chlorophenyl)-4-(1-cyclohexylazetidin-3-yloxy)-2-methylpyrazolo[1,5-a][1,3,5]triazine;
7-(2-chlorophenyl)-8-(4-chlorophenyl )-4-isopropoxy-2-methylpyrazolo[1,5-a][1,3,5]triazine; and
4-tert-butoxy-7-(2-chlorophenyl)-8-(4-chlorophenyl)-2-methylpyrazolo[1,5-a][1,3,5]triazine;
butyl-[7-(2-chlorophenyl)-8-(4-chlorophenyl)-2-methylpyrazolo[1,5-a][1,3,5]triazin-4-yl]-amine;
7-(2-chlorophenyl)-8-(4-chlorophenyl)-2-methyl-4-piperidin-1-yl-pyrazolo[1,5-a][1,3,5]triazine;
[7-(2-chlorophenyl)-8-(4-chlorophenyl)-2-methylpyrazolo[1,5-a][1,3,5]triazin-4-yl]-[2-(4-fluorophenyl)-ethyl]-amine;
7-(2-chlorophenyl)-8-(4-chlorophenyl)-2-methyl-4-morpholin-4-yl-pyrazolo[1,5-a][1,3,5]triazine; and
[7-(2-chlorophenyl)-8-(4-chlorophenyl)-2-methylpyrazolo[1,5-a][1,3,5]triazin-4-yl]-(2-morpholin-4-yl-ethyl)-amine;
1-[7-(2-chlorophenyl)-8-(4-chlorophenyl)-2-methylpyrazolo[1,5-a][1,3,5]triazin-4-yl]-4-ethylaminopiperidine-4-carboxylic acid amide;
1-[7-(2-chlorophenyl)-8-(4-chlorophenyl)-2-methylpyrazolo[1,5-a][1,3,5]triazin-4-yl]-3-ethylaminoazetidine-3-carboxylic acid amide;
1-[7-(2-chlorophenyl)-8-(4-chlorophenyl)-2-methylpyrazolo[1,5-a][1,3,5]triazin-4-yl]-3-isopropylaminoazetidine-3-carboxylic acid amide;
3-amino-1-[7-(2-chlorophenyl)-8-(4-chlorophenyl)-2-methylpyrazolo[1,5-a][1,3,5]triazin-4-yl]-azetidine-3-carboxylic acid amide; and
8-[7-(2-chlorophenyl)-8-(4-chlorophenyl)-2-methylpyrazolo[1,5-a][1,3,5]triazin-4-yl]-1-isopropyl-1,3,8-triazaspiro[4.5]decan-4-one; and
a pharmaceutically acceptable salt thereof or a solvate or hydrate of said compound or said salt.

15. The method according to claim 12, wherein said CB-1 receptor antagonist is selected from:
3-(4-chlorophenyl)-2-(2-chlorophenyl)-7-(4-methyl-piperazin-1-yl)-pyrazolo[1,5-a]pyrimidine;
3-(4-chlorophenyl)-2-(2-chlorophenyl)-7-(4-pyrimidin-2-yl-piperazin-1-yl)-pyrazolo[1,5-a]pyrimidine;
3-(4-chloro-phenyl)-2-(2-chlorophenyl)-7-[(1S,4S)-5-methanesulfonyl-2,5-diazabicyclo[2.2.1]hept-2-yl]-pyrazolo[1,5-a]pyrimidine; and
3-(4-chlorophenyl)-2-(2-chlorophenyl)-7-[4-(propane-2-sulfonyl)-piperazin-1-yl]-pyrazolo[1,5-a]pyrimidine;
1-[3-(4-chlorophenyl)-2-(2-chlorophenyl)-pyrazolo[1,5-a]pyrimidin-7-yl]-4-ethylaminopiperidine-4-carboxylic acid amide;
1-[3-(4-chlorophenyl)-2-(2-chlorophenyl)-pyrazolo[1,5-a]pyrimidin-7-yl]-4-isopropylaminopiperidine-4-carboxylic acid amide;
1-[3-(4-chlorophenyl)-2-(2-chlorophenyl)-pyrazolo[1,5-a]pyrimidin-7-yl]-3-ethylaminoazetidine-3-carboxylic acid amide;
3-amino-1-[3-(4-chlorophenyl)-2-(2-chlorophenyl)-pyrazolo[1,5-a]pyrimidin-7-yl]-azetidine-3-carboxylic acid amide;
1-[3-(4-chlorophenyl)-2-(2-chlorophenyl)-6-methylpyrazolo[1,5-a]pyrimidin-7-yl]-3-ethylaminoazetidine-3-carboxylic acid amide;
1-[3-(4-chlorophenyl)-2-(2-chlorophenyl)-pyrazolo[1,5-a]pyrimidin-7-yl]-3-isopropylaminoazetidine-3-carboxylic acid amide;
1-[3-(4-chlorophenyl)-2-(2-chlorophenyl)-5,6-dimethylpyrazolo[1,5-a]pyrimidin-7-yl]-3-ethylamino-azetidine-3-carboxylic acid amide;
1-[3-(4-chlorophenyl)-2-(2-chlorophenyl)-pyrazolo[1,5-a]pyrimidin-7-yl]-3-methylaminoazetidine-3-carboxylic acid amide;
1-[3-(4-chlorophenyl)-2-(2-chlorophenyl)-5-methylpyrazolo[1,5-a]pyrimidin-7-yl]-3-ethylaminoazetidine-3-carboxylic acid amide;
1-{1 -[3-(4-chlorophenyl)-2-(2-chlorophenyl)-pyrazolo[1,5-a]pyrimidin-7-yl]-4-phenylpiperidin-4-yl}-ethanone;
3-[3-(4-chlorophenyl)-2-(2-chlorophenyl)-pyrazolo[1,5-a]pyrimidin-7-yl]-3-(1a,5a,6a)-azabicyclo[3.1.0]hex-6-ylamine;
1-[3-(4-chlorophenyl)-2-(2-chlorophenyl)-pyrazolo[1,5-a]pyrimidin-7-yl]-4-(4-fluorophenyl)-piperidin-4-ol;
4-benzyl-1-[3-(4-chlorophenyl)-2-(2-chlorophenyl)-pyrazolo[1,5-a]pyrimidin-7-yl]-piperidin-4-ol;
8-[3-(4-chlorophenyl)-2-(2-chlorophenyl)-pyrazolo[1,5-a]pyrimidin-7-yl]-1-isopropyl-1,3,8-triazaspiro[4.5]decan-4-one;
2-[3-(4-chlorophenyl)-2-(2-chlorophenyl)-pyrazolo[1,5-a]pyrimidin-7-yl]-2,5,7-triazaspiro[3.4]octan-8-one;
8-[3-(4-chlorophenyl)-2-(2-chlorophenyl)-6-methylpyrazolo[1,5-a]pyrimidin-7-yl]-1-isopropyl-1,3,8-triazaspiro[4.5]decan-4-one;
2-[3-(4-chlorophenyl)-2-(2-chlorophenyl)-pyrazolo[1,5-a]pyrimidin-7-yl]-5-methyl-2,5,7-triazaspiro[3.4]octan-8-one;
7-(1-benzylpyrrolidin-3-yloxy)-3-(4-chlorophenyl)-2-(2-chlorophenyl)-pyrazolo[1,5-a]pyrimidine; and
3-(4-chlorophenyl)-2-(2-chlorophenyl)-7-(1-cyclohexylazetidin-3-yloxy)-pyrazolo[1,5-a]pyrimidine;
1-[3-(4-chlorophenyl)-2-(2-chlorophenyl)-pyrazolo[1,5-a]pyrimidin-7-yl]-4-ethylaminopiperidine-4-carboxylic acid amide;
1-[3-(4-chlorophenyl)-2-(2-chlorophenyl)-pyrazolo[1,5-a]pyrimidin-7-yl]-4-isopropylaminopiperidine-4-carboxylic acid amide; and
1-[3-(4-chlorophenyl)-2-(2-chlorophenyl)-pyrazolo[1,5-a]pyrimidin-7-yl]-3-ethylaminoazetidine-3-carboxylic acid amide; and
a pharmaceutically acceptable salt thereof or a solvate or hydrate of said compound or said salt.

16. The method according to claim 12, wherein said CB-1 receptor antagonist is selected from:
5-(4-chloro-phenyl)-3-(5-cyclohexyl-1H-imidazol-2-yl)-1-(2,4-dichloro-phenyl)-4-methyl-1H-pyrazole;
5-(4-chloro-phenyl)-3-(2-cyclohexyl-3H-imidazol-4-yl)-1-(2,4-dichloro-phenyl)-4-methyl-1H-pyrazole;
5-(4-chloro-phenyl)-1-(2,4-dichloro-phenyl)-4-methyl-3-[1-(1-methyl-1-phenyl-ethyl)-1H-imidazol-4-yl]-1H-pyrazole;
5-(4-chloro-phenyl)-1-(2-chloro-phenyl)-4-methyl-3-[1-(1-phenyl-ethyl)-1H-imidazol-4-yl]-1H-pyrazole;
5-(4-chloro-phenyl)-1-(2-fluoro-phenyl)-4-methyl-3-[1-(1-methyl-1-phenyl-ethyl)-1H-imidazol-4-yl]-1H-pyrazole;
5-(4-chloro-phenyl)-1-(2-chloro-phenyl)-3-[1-(2,2-dimethyl-tetrahydro-pyran-4-yl )-1H-imidazol-4-yl]-4-methyl-1H-pyrazole:
5-{2-(2,4-dichloro-phenyl)-4-methyl-5-[1-(1-methyl-1-phenyl-ethyl)-1H-imidazol-4-yl]-2H-pyrazol-3-yl}-2-methoxy-pyridine; and
1-(2-chloro-phenyl)-5-(4-chloro-phenyl)-4-methyl-3-[1-(1-methyl-1-phenyl-ethyl)-1H-imidazol-4-yl]-1H-pyrazole; and
a pharmaceutically acceptable salt thereof or a solvate or hydrate of the compound or the salt.

17. The method according to claim 12, wherein said CB-1 receptor antagonist is selected from:
1-[5-(4-chloro-phenyl)-1-(2-chloro-phenyl)-4-methyl-1H-pyrazol-3-yl]-2-piperidin-1-yl-ethanone;
1-[5-(4-chloro-phenyl)-1-(2-chloro-phenyl)-4-methyl-1H-pyrazol-3-yl]-2-morpholin-4-yl-ethanone;
1-[5-(4-chloro-phenyl)-1-(2-chloro-phenyl)-4-methyl-1H-pyrazol-3-yl]-2-[4-(1-methyl-1H-pyrrole-2-carbonyl)-piperazin-1-yl]-ethanone;
1-[5-(4-chloro-phenyl)-1-(2-chloro-phenyl)-4-methyl-1H-pyrazol-3-yl]-2-[4-(1-methyl-cyclopropanecarbonyl)-piperazin-1-yl]-ethanone;
N-(1-{2-[5-(4-chloro-phenyl)-1-(2-chloro-phenyl)-4-methyl-1H-pyrazol-3-yl]-2-oxo-ethyl}-piperidin-4-yl)-2,2,2-trifluoro-acetamide;
1-[5-(4-chloro-phenyl)-1-(2-fluoro-phenyl )-4-methyl- 1H-pyrazol-3-yl]-2-morpholin-4-yl-ethanone;
1-[5-(4-chloro-phenyl)-1-(2-fluoro-phenyl)-4-methyl-1H-pyrazol-3-yl]-2-piperidin-1-yl-ethanone;
1-[5-(4-chloro-phenyl)-1-(2-fluoro-phenyl)-4-methyl-1H-pyrazol-3-yl]-2-(4-trifluoroacetyl-piperazin-1-yl)-ethanone;
1-[1 -(2-chloro-phenyl)-5-(4-chloro-phenyl)-4-methyl-1H-pyrazol-3-yl]-2-pyrrolidin-1-yl-thanone;
1-[1 -(2-chloro-phenyl)-5-(4-chloro-phenyl)-4-methyl-1H-pyrazol-3-yl]-2-[1,4]oxazepan-4-yl-ethanone; and
1-[5-(4-chloro-phenyl)-1-(2-chloro-phenyl)-4-methyl-1H-pyrazol-3-yl]-2-(1-oxa-8-aza-spiro[4.5]dec-8-yl)-ethanone; and
a pharmaceutically acceptable salt thereof, or a solvate or hydrate of the compound or the salt.

18. The method according claim 12, wherein said CB-1 receptor antagonist is selected from:
2-(benzyl-isopropyl-amino)-1-[1 -(2-chloro-phenyl)-5-(4-chloro-phenyl)-4-methyl-1H-pyrazol-3-yl]-ethanol;
1-[5-(4-chloro-phenyl)-1-(2-chloro-phenyl)-4-methyl-1H-pyrazol-3-yl]-2-(3,5-dimethyl-piperidin-1-yl)-ethanol;
1-{2-[1-(2-chloro-phenyl)-5-(4-chloro-phenyl)-4-methyl-1H-pyrazol-3-yl]-2-hydroxy-ethyl}-4-isopropylamino-piperidine-4-carboxylic acid amide;
1-[5-(4-chloro-phenyl)-1-(2-chloro-phenyl)-4-methyl-1H-pyrazol-3-yl]-2-(3,3-dimethyl-piperidin-1-yl)-ethanol;
1-[5-(4-chloro-phenyl)-1-(2-chloro-phenyl)-4-methyl-1H-pyrazol-3-yl]-2-piperidin-1-yl-ethanol; and
1-[5-(4-chloro-phenyl)-1-(2-chloro-phenyl)-4-methyl-1H-pyrazol-3-yl]-2-morpholin-4-yl-ethanol; and
a pharmaceutically acceptable salt thereof, or a solvate or hydrate of the compound or the salt.

19. The method according to claim 12 wherein said CB-1 receptor antagonist is selected from:
2-[5-(4-chloro-phenyl)-1-(2-chloro-phenyl)-4-methyl-1H-pyrazol-3-yl]-4-cyclohexyl-morpholine;
2-[5-(4-chloro-phenyl)-1-(2-chloro-phenyl)-4-methyl-1H-pyrazol-3-yl]-4-(propane-2-sulfonyl)-morpholine;
2-[5-(4-chloro-phenyl)-1-(2-chloro-phenyl)-4-methyl-1H-pyrazol-3-yl]-4-(toluene-4-sulfonyl)-morpholine;
1-{2-[1-(2-chloro-phenyl)-5-(4-chloro-phenyl)-4-methyl-1H-pyrazol-3-yl]-morpholin-4-yl}-2-methyl-propan-1-one; and
2-[1-(2-chloro-phenyl)-5-(4-chloro-phenyl)-4-methyl-1H-pyrazol-3-yl]-4-(4-trifluoromethyl-benzyl)-morpholine; and
a pharmaceutically acceptable salt thereof or a solvate or hydrate of the compound or the salt.

20. The method according claim 12, wherein said CB-1 receptor antagonist is selected from:
1-[1-(4-chloro-phenyl)-2-(2,4-dichloro-phenyl)-5-methyl-1H-imidazol-4-yl]-2-piperidin-1-yl-ethanone and
1-[1-(4-chloro-phenyl)-2-(2,4-dichloro-phenyl)-5-methyl-1H-imidazol-4-yl]-2-morpholin-4-yl-ethanone; and
a pharmaceutically acceptable salt thereof, a or a solvate or hydrate of the compound, or the salt.

21. The method according to claim 12, wherein the opioid receptor antagonist is selected from:
2-methoxy-ethanesulfonic acid {3-[6-ethyl-3-(2-hydroxy-1,2,3,4-tetrahydro-naphthalen-2-ylmethyl)-3-aza-bicyclo[3.1.0]hex-6-yl]-phenyl}-amide;
N-[3-(6-ethyl-3-indan-2-ylmethyl-3-aza-bicyclo[3.1.0]hex-6-yl )-phenyl]-methanesulfonamide;
2-methoxy-ethanesulfonic acid [3-(6-ethyl-3-indan-2-ylmethyl-3-aza-bicyclo[3.1.0]hex-6-yl)-phenyl]-amide;
N-{3-[6-ethyl-3-(2-hydroxy-indan-2-yl methyl )-3-aza-bicyclo[3.1.0]hex-6-yl]-phenyl}-methanesulfonamide;
N-{3-[6-ethyl-3-(2-hydroxy-1,2,3,4-tetrahydro-naphthalen-2-ylmethyl )-3-aza-bicyclo[3.1.0]hex-6-yl]-phenyl}-methanesulfonamide;
N-(3-{6-ethyl-3-[3-(1-hydroxy-cyclohexyl )-propyl]-3-aza-bicyclo[3.1.0]hex-6-yl}-phenyl)-methanesulfonamide;
3-{3-[3-(1-hydroxy-cyclohexyl)-propyl]-8-methoxy-3-aza-bicyclo[3.2.1]oct-8-yl}-benzamide;
2-methoxy-ethanesulfonic acid {3-[6-ethyl-3-(2-hydroxy-indan-2-ylmethyl)-3-aza-bicyclo[3.1.0]hex-6-yl]-phenyl}-amide;
3-[6-ethyl-3-(2-hydroxy-indan-2-ylmethyl)-3-aza-bicyclo[3.1.0]hex-6-yl]-benzamide;
N-{3-[6-ethyl-3-(2-hydroxy-1,2,3,4-tetrahydro-naphthalen-2-ylmethyl )-3-aza-bicyclo[3.1.0]hex-6-yl]-phenyl}-methanesulfonamide;
3-[6-ethyl-3-(2-hydroxy-1,2,3,4-tetrahydro-naphthalen-2-ylmethyl)-3-aza-bicyclo[3.1.0]hex-6-yl]-benzamide;
N-{3-[2-(2-hydroxy-indan-2-ylmethyl)-2-aza-bicyclo[3.3.1]non-5-yl]-phenyl}-methanesulfonamide;
2-methoxy-ethanesulfonic acid {3-[3-(2-hydroxy-indan-2-ylmethyl)-8-methoxy-3-aza-bicyclo[3.2.1]oct-8-yl]-phenyl}-amide;
3-{6-ethyl-3-[3-(1-hydroxy-cyclohexyl)-propyl]-3-aza-bicyclo[3.1.0]hex-6-yl}-benzamide;
3-[6-ethyl-3-(1-hydroxy-3-phenyl-cyclobutylmethyl)-3-aza-bicyclo[3.1.0]hex-6-yl]-benzamide;
2-methoxy-ethanesulfonic acid (3-{6-ethyl-3-[3-(1-hydroxy-cyclohexyl)-propyl]-3-aza-bicyclo[3.1.0]hex-6-yl}-phenyl)-amide;
3-{1 -[3-(1-hydroxy-cyclohexyl)-propyl]-3,4-dimethyl-piperidin-4-yl}-benzamide;
3-(1-indan-2-ylmethyl-3,4-dimethyl-piperidin-4-yl)-benzamide;
N-{3-[3-(2-hydroxy-indan-2-ylmethyl)-8-methoxy-3-aza-bicyclo[3.2.1]oct-8-yl]-phenyl}-methanesulfonamide;
3-[1 -(1-hydroxy-3-phenyl-cyclobutylmethyl)-3,4-dimethyl-piperidin-4-yl]-benzamide;
3-(6-hthyl-3-indan-2-ylmethyl-3-aza-bicyclo[3.1.0]hex-6-yl)-benzamide;
N-(3-{3-[3-(1-hydroxy-cyclohexyl)-propyl]-8-methoxy-3-aza-bicyclo[3.2.1]oct-8-yl}-phenyl)-methanesulfonamide;
3-[1 -(2-hydroxy-indan-2-ylmethyl)-3,4-dimethyl-piperidin-4-yl]-benzamide;
N-(3-{2-[3-(1-hydroxy-cyclohexyl)-propyl]-2-aza-bicyclo[3.3.1]non-5-yl}-phenyl)-methanesulfonamide;
3-[3-(1-hydroxy-3-phenyl-cyclobutylmethyl)-8-methoxy-3-aza-bicyclo[3.2.1]oct-8-yl]-benzamide;
3-[3-(2-hydroxy-indan-2-ylmethyl)-8-methoxy-3-aza-bicyclo[3.2.1]oct-8-yl]-benzamide;
3-[2-(2-hydroxy-indan-2-yl methyl)-2-aza-bicyclo[3.3.1]non-5-yl]-benzamide;
3-{2-[3-(1-hydroxy-cyclohexyl)-propyl]-2-aza-bicyclo[3.3.1]non-5-yl}-benzamide;
3-[2-(1-hydroxy-3-phenyl-cyclobutylmethyl)-2-aza-bicyclo[3.3.1]non-5-yl]-benzamide;
3-[2-(2-hydroxy-1,2,3,4-tetrahydro-naphthalen-2-ylmethyl)-2-aza-bicyclo[3.3.1]non-5-yl]-benzamide;
2-methoxy-ethanesulfonic acid {3-[2-(2-hydroxy-indan-2-ylmethyl)-2-aza-bicyclo[3.3.1]non-5-yl]-phenyl}-amide;
2-methoxy-ethanesulfonic acid (3-{2-[3-(1-hydroxy-cyclohexyl)-propyl]-2-aza-bicyclo[3.3.1]non-5-yl}-phenyl)-amide;
2-methoxy-ethanesulfonic acid {3-[2-(1-hydroxy-3-phenyl-cyclobutylmethyl)-2-aza-bicyclo[3.3.1]non-5-yl]-phenyl}-amide;
2-methoxy-ethanesulfonic acid {3-[2-(2-hydroxy-1,2,3,4-tetrahydro-naphthalen-2-ylmethyl)-2-aza-bicyclo[3.3.1]non-5-yl]-phenyl}-amide;
N-{3-[2-(1-hydroxy-3-phenyl-cyclobutylmethyl)-2-aza-bicyclo[3.3.1]non-5-yl]-phenyl}-methanesulfonamide; and
N-{3-[2-(2-hydroxy-1,2,3,4-tetrahydro-naphthalen-2-ylmethyl)-2-aza-bicyclo[3.3.1]non-5-yl]-phenyl}-methanesulfonamide.

22. The method according to claim 12, wherein the opioid receptor antagonist is selected from:
2-methoxy-ethanesulfonic acid {3-[6-ethyl-3-(2-hydroxy-1,2,3,4-tetrahydro-naphthalen-2-ylmethyl)-3-aza-bicyclo[3.1.0]hex-6-yl]-phenyl}-amide;
N-[3-(6-ethyl-3-indan-2-ylmethyl-3-aza-bicyclo[3.1.0]hex-6-yl)-phenyl]-methanesulfonamide;
2-methoxy-ethanesulfonic acid [3-(6-ethyl-3-indan-2-yl methyl-3-aza-bicyclo[3.1.0]hex-6-yl)-phenyl]-amide;
N-{3-[6-ethyl-3-(2-hydroxy-indan-2-ylmethyl)-3-aza-bicyclo[3.1.0]hex-6-yl]-phenyl}-methanesulfonamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
N-(3-{6-ethyl-3-[3-(1-hydroxy-cyclohexyl)-propyl]-3-aza-bicyclo[3.1.0]hex-6-yl}-phenyl)-methanesulfonamide;
3-{3-[3-(1-hydroxy-cyclohexyl)-propyl]-8-methoxy-3-aza-bicyclo[3.2.1]oct-8-yl}-benzamide;
2-methoxy-ethanesulfonic acid {3-[6-ethyl-3-(2-hydroxy-indan-2-ylmethyl )-3-aza-bicyclo[3.1.0]hex-6-yl]-phenyl}-amide;
3-[6-ethyl-3-(2-hydroxy-indan-2-ylmethyl)-3-aza-bicyclo[3.1.0]hex-6-yl]-benzamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
3-[6-ethyl-3-(2-hydroxy-1,2,3,4-tetrahydro-naphthalen-2-ylmethyl)-3-aza-bicyclo[3.1.0]hex-6-yl]-benzamide;
N-{3-[2-(2-hydroxy-indan-2-ylmethyl)-2-aza-bicyclo[3.3.1]non-5-yl]-phenyl}-methanesulfonamide;
2-methoxy-ethanesulfonic acid {3-[3-(2-hydroxy-indan-2-ylmethyl)-8-methoxy-3-aza-bicyclo[3.2.1]oct-8-yl]-phenyl}-amide;
3-{6-ethyl-3-[3-(1-hydroxy-cyclohexyl)-propyl]-3-aza-bicyclo[3.1.0]hex-6-yl}-benzamide;
3-[6-ethyl-3-(1-hydroxy-3-phenyl-cyclobutylmethyl)-3-aza-bicyclo[3.1.0]hex-6-yl]-benzamide;
2-methoxy-ethanesulfonic acid (3-{6-ethyl-3-[3-(1-hydroxy-cyclohexyl)-propyl]-3-aza-bicyclo[3.1.0]hex-6-yl}-phenyl)-amide;
3-{1 -[3-(1-hydroxy-cyclohexyl)-propyl]-3,4-dimethyl-piperidin-4-yl}-benzamide;
3-(1-indan-2-ylmethyl-3,4-dimethyl-piperidin-4-yl)-benzamide;
N-{3-[3-(2-hydroxy-indan-2-ylmethyl)-8-methoxy-3-aza-bicyclo[3.2.1]oct-8-yl]-phenyl}-methanesulfonamide;
3-[1 -(1-hydroxy-3-phenyl-cyclobutylmethyl)-3,4-dimethyl-piperidin-4-yl]-benzamide;
3-(6-hthyl-3-indan-2-ylmethyl-3-aza-bicyclo[3.1.0]hex-6-yl)-benzamide;
N-(3-{3-[3-(1-hydroxy-cyclohexyl)-propyl]-8-methoxy-3-aza-bicyclo[3.2.1]oct-8-yl}-phenyl)-methanesulfonamide;
3-[1 -(2-hydroxy-indan-2-ylmethyl)-3,4-dimethyl-piperidin-4-yl]-benzamide;
N-(3-{2-[3-(1-hydroxy-cyclohexyl)-propyl]-2-aza-bicyclo[3.3.1]non-5-yl}-phenyl)-methanesulfonamide;
3-[3-(1-hydroxy-3-phenyl-cyclobutylmethyl)-8-methoxy-3-aza-bicyclo[3.2.1]oct-8-yl]-benzamide;
3-[3-(2-hydroxy-indan-2-yl methyl)-8-methoxy-3-aza-bicyclo[3.2.1]oct-8-yl]-benzamide;
3-[2-(2-hydroxy-indan-2-yl methyl)-2-aza-bicyclo[3.3.1]non-5-yl]-benzamide;
3-{2-[3-(1-hydroxy-cyclohexyl)-propyl]-2-aza-bicyclo[3.3.1]non-5-yl}-benzamide;
3-[2-(1-hydroxy-3-phenyl-cyclobutylmethyl)-2-aza-bicyclo[3.3.1]non-5-yl]-benzamide;
3-[2-(2-hydroxy-1,2,3,4-tetrahydro-naphthalen-2-ylmethyl)-2-aza-bicyclo[3.3.1]non-5-yl]-benzamide;
2-methoxy-ethanesulfonic acid {3-[2-(2-hydroxy-indan-2-ylmethyl)-2-aza-bicyclo[3.3.1]non-5-yl]-phenyl}-amide;
2-methoxy-ethanesulfonic acid (3-{2-[3-(1-hydroxy-cyclohexyl)-propyl]-2-aza-bicyclo[3.3.1]non-5-yl}-phenyl)-amide;
2-methoxy-ethanesulfonic acid {3-[2-(1-hydroxy-3-phenyl-cyclobutylmethyl)-2-aza-bicyclo[3.3.1]non-5-yl]-phenyl}-amide;
2-methoxy-ethanesulfonic acid {3-[2-(2-hydroxy-1,2,3,4-tetrahydro-naphthalen-2-ylmethyl)-2-aza-bicyclo[3.3.1]non-5-yl]-phenyl}-amide;
N-{3-[2-(1-hydroxy-3-phenyl-cyclobutylmethyl)-2-aza-bicyclo[3.3.1]non-5-yl]-phenyl}-methanesulfonamide; and
N-{3-[2-(2-hydroxy-1,2,3,4-tetrahydro-naphthalen-2-ylmethyl)-2-aza-bicyclo[3.3.1]non-5-yl]-phenyl}-methanesulfonamide.

23. The method according to claim 12, wherein the opioid receptor antagonist and the CB-1 receptor antagonist are administered substantially simultaneously.
